(19) United States
(12) Patent Application Publication
(10) Pub. No.: US 2022/0309042 A1
(43) Pub. Date: Sep. 29, 2022

(54) LOCAL EXERCISE DEVICE NETWORK

(71) Applicant: iFIT Inc., Logan, UT (US)

(72) Inventor: Mark Archer, Cathedral City, CA (US)

(21) Appl. No.: 17/702,021

(22) Filed: Mar. 23, 2022

Related U.S. Application Data
(60) Provisional application No. 63/165,498, filed on Mar. 24, 2021.

(51) Int. Cl. G06F 16/182; G06F 16/178; H04L 67/1087; H04L 67/06

(52) U.S. Cl. CPC G06F 16/1834; G06F 16/178; H04L 67/1091; H04L 67/06

(57) ABSTRACT

One or more exercise devices may be connected to a local exercise device network. A user may select an exercise program to be performed that not located on the user's exercise device. Before downloading the exercise program from the cloud, the exercise device receives the requested exercise program from the other exercise devices on the exercise device network.

### CROSS-REFERENCE TO RELATED APPLICATIONS

This application claims priority to and the benefit of United States Provisional Patent Application No. 63/165,498, filed on Mar. 24, 2021, which are hereby incorporated by reference in its entirety.

### BACKGROUND

### Background and Relevant Art

Modern exercise devices often include pre-recorded exercise programs. An exercise program may include instructions for the user to follow while performing the exercise. The instructions may include pre-determined resistance, incline, speed, or other exercise device parameters. Some exercise devices may include a console having a display. While performing the exercise program, the display may present instruction, motivation, entertainment, user exercise data, any other information, and combinations thereof.

Many exercise programs may include pre-recorded content to be presented on the display. The pre-recorded content may include videos of trainers, motivational videos, graphics, music, sound effects, audio overlay, entertainment, and so forth. The pre-recorded content may be recorded as a video or multi-media file. The pre-recorded content may be stored in a remote server (e.g., the cloud). The exercise device may download the pre-recorded content from the Internet.

Hosting content on a remote server may include access and use fees. The host may charge one or more fees for the number of times the remote server is accessed and/or for the total amount of data downloaded from the remote server.

### BRIEF SUMMARY

In some embodiments, a method for implementing an exercise program includes receiving an input on a first exercise device to perform the exercise program. The exercise program is part of a local exercise device network including a plurality of exercise devices. The method includes determining whether the exercise program is located on another network device on the exercise device network. If the data file is on the other exercise device, the exercise program is transferred to the exercise device over the exercise device network. If the data file is not located on any network device, the data file is downloaded from a remote server.

In some embodiments, an exercise system includes a first exercise device and a second exercise device. The exercise devices include local memory having one or more exercise programs. The exercise devices are in connection using communication systems in direct communication with each other.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF THE DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 is a representation of an exercise device network, according to at least one embodiment of the present disclosure;

FIG. 2 is a representation of an exercise device network including a local server, according to at least one embodiment of the present disclosure;

FIG. 3 is a representation of a network exercise device, according to at least one embodiment of the present disclosure;

FIG. 4 is a representation of the local storage of an exercise device network, according to at least one embodiment of the present disclosure;

FIG. 5 is a flowchart of a method for implementing an exercise program, according to at least one embodiment of the present disclosure;

FIG. 6 is a representation of the local storage of another exercise device network, according to at least one embodiment of the present disclosure;

FIG. 7 is a flowchart of another method for implementing an exercise program, according to at least one embodiment of the present disclosure;

FIG. 8 is a representation of another exercise device network, according to at least one embodiment of the present disclosure;

FIG. 9 is a flowchart of another method for implementing an exercise program, according to at least one embodiment of the present disclosure;

FIG. 10 is a flowchart of another method for implementing an exercise program, according to at least one embodiment of the present disclosure;

FIG. 11 is a representation of a schedule for an exercise device network, according to at least one embodiment of the present disclosure; and

FIG. 12 is a representation of another exercise program, according to at least one embodiment of the present disclosure.

### DETAILED DESCRIPTION

This disclosure generally relates to devices, systems, and methods for creating a peer to peer (P2P) network between exercise devices. When a user selects a new exercise program to perform on an exercise device, the exercise device may look for the exercise program in the memory of other exercise devices connected to the network. If another connected exercise device has the exercise program stored in memory, then the connected exercise device may transmit the exercise program to the user's exercise device over the local network. In this manner, the number of times that the exercise program is downloaded from a cloud server may be decreased. This may increase the download speed to the user's exercise device and/or reduce server fees caused by the transmission of the exercise program over the Internet.

Using the P2P network, a user may experience a seamless transition between different exercise devices in the same workout. Each exercise device may track exercise data about the user, such as distance, heart rate, calories, time exercising, transition times, and so forth. The exercise devices may communicate the exercise data between each other. The exercise network may further prepare upcoming exercise devices for the user to use next. The user's next scheduled exercise device to use may be indicated to the user, and the next exercise device reserved for the user's use. This may reduce the amount of unnecessary and unwanted downtime during the exercise program. This may improve the exercise experience by providing a seamless transition between exercises.

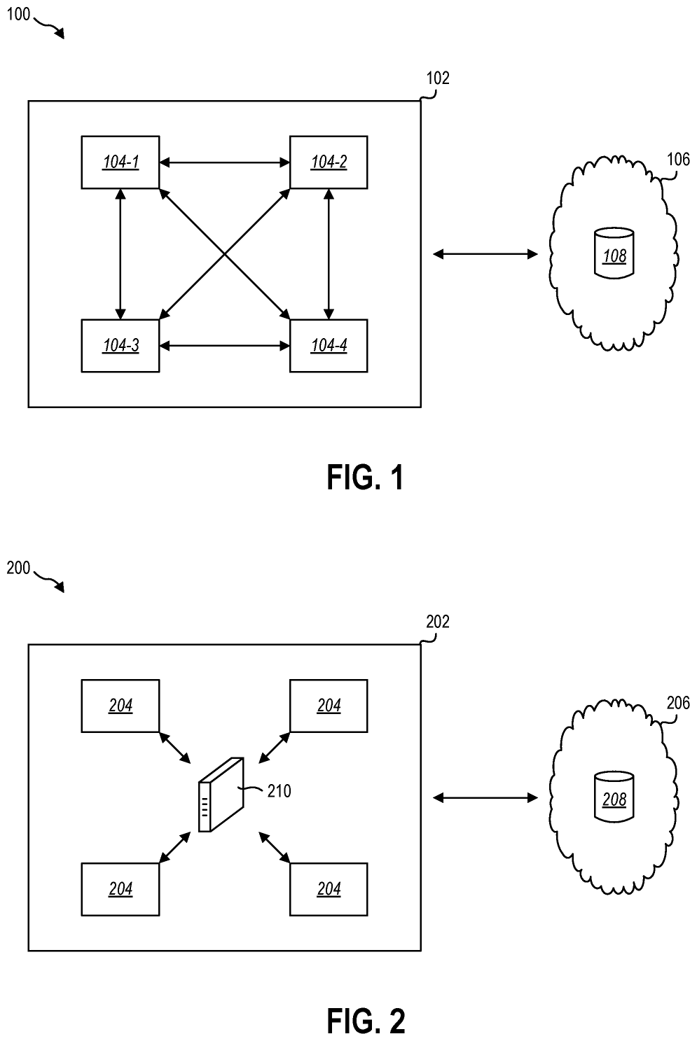

FIG. 1 is a representation of an exercise device network 100, according to at least one embodiment of the present disclosure. The exercise device network 100 includes a local network 102. The local network 102 includes two or more interconnected exercise devices (collectively 104). In some embodiments, the exercise devices 104 may include any type of exercise device. For example, the exercise devices 104 may include a treadmill, a stationary bicycle, an elliptical, a rower, a cable resistance trainer, a weight machine, free weights, smart weights (e.g., free weights including one or more sensor connected to the local network 102) any other type of exercise device, and combinations thereof.

In some embodiments, each exercise device 104 in the local network 102 may be the same type of exercise device. For example, each exercise device 104 may be a treadmill. In some embodiments, each exercise device 104 in the local network 102 may be a different type of exercise device. For example, a first exercise device 104-1 may be a treadmill, a second exercise device 104-2 may be a stationary bicycle, a third exercise device 104-3 may be a rower, and a fourth exercise device 104-4 may be a set of smart weights. In some embodiments, the local network 102 may include multiple exercise devices 104. For example, the first exercise device 104-1 and the second exercise device 104-2 may be treadmills, and the third exercise device 104-3 and the fourth exercise device 104-4 may be stationary bicycles. In some embodiments, the local network 102 may include any combination of exercise device types.

While, in the embodiment shown, the local network 102 includes four exercise devices 104, it should be understood that the local network 102 may include any number of exercise devices 104, including 2, 3, 4, 5, 6, 7, 8, 9, 10, 15, 20, 25, 30, 40, 50, 75, 100, or more exercise devices 104. For example, the local network 102 may be part of a commercial gym, and the exercise devices 104 may all be part of the same gym. In some examples, the local network 102 may be part of a home gym, and the exercise devices 104 may all be part of the user's home gym. In some examples, the local network 102 may include two or more home gyms connected over the Internet. Connecting multiple home gyms over the Internet may reduce the amount of data transferred over a remote server 106, thereby reducing any data fees associated with access to the remote server 106.

In some embodiments, the exercise devices 104 may be connected over a local area network (LAN). In some examples, the exercise devices 104 may be wirelessly connected over a wireless network, such as a Wi-Fi network (e.g., IEEE 802.11 family of standards). In some examples, the exercise devices 104 may be connected wirelessly in another manner, such as via Bluetooth, Z-wave technology, Zigbee protocol, any other wireless connection, and combinations thereof. In some examples, the LAN may include one or more Internet connections. In some examples, the LAN may include one or more Internet connections that do not connect to a remote server 106. In some examples, the exercise devices 104 may be connected over a wired connection, such as an ethernet cable.

In accordance with embodiments of the present disclosure, the exercise devices 104 may be in communication with each other. For example, the exercise devices 104 may send and receive information between each other. In some embodiments, the exercise devices 104 may send and receive information over the local network 102. In some embodiments, the exercise devices 104 may send and receive information over the local network 102 without the information being uploaded and/or downloaded from a remote server 106. In some embodiments, the exercise devices 104 may send and receive information over the local network 102 without the information being uploaded and/or downloaded from the remote server 106. In some embodiments, the exercise devices 104 may send and receive information over the local network 102 without sending the information over the Internet. Put another way, information may be transferred across the local network 102 without a remote server (e.g., the remote server 106) negotiating the communication. Data transfer may happen without an intermediate server. For example, access to a remote server 106 may be controlled by a single router or access point. All data transfer with the remote server 106 may be controlled through the single router or access point. In this manner, a local network 102 may transfer information between devices within the local network 102 without transmitting information through the single router or access point.

In some embodiments, the remote server 106 may be physically close but not connected to the local network 102. In some embodiments, the remote server 106 may be physically distant from the local network 102. For example, the remote server 106 may be located in a different city, in a different state/province, in a different country, in a different continent, or any other remote location. In some embodiments, data transfer across geographical boundaries (cities, states/provinces, countries, continents) may increase access costs to the remote server 106 and/or decrease data file transfer feed. In this manner, transmitting information (such as exercise programs) across the local network 102 (e.g., not from the remote server 106) may help to decrease access costs and increase transmission speed of information across the local network 102.

In some embodiments, the local network 102 may be considered a peer to peer (P2P) network. A P2P network may be any network in which the computing elements of exercise devices 104 are in communication with other computing elements of exercise devices 104 within the same network. Exercise devices 104 within the same P2P network may communicate with and transfer data between computing devices. In some embodiments, such as the embodiment shown in FIG. 1, each exercise device 104 within a P2P network may be in direct communication with each other exercise device. In some embodiments, two exercise devices may be in indirect communication with each other, such as in an indirect mesh network. For example, the first exercise device 104-1 may be in communication with the fourth exercise device 104-4 indirectly through the third exercise device 104-3. Thus, the fourth exercise device 104-4 may transmit information to the first exercise device 104-1 by first transmitting the information to the third exercise device 104-3, and the third exercise device 104-3 may then transmit the information to the first exercise device 104-1. Thus, the third exercise device 104-3 may act as a relay between the fourth exercise device 104-4 and the first exercise device 104-1.

The local network 102 may be connected to the remote server 106 over the Internet. The remote server 106 may include a database 108 of exercise programs. Each of the exercise devices 104 may be in communication with the remote server 106. When preparing to exercise, a user may select an exercise program from a list of available exercise programs to perform on the first exercise device 104-1. Conventionally, if the selected exercise program is not stored locally on the first exercise device 104-1, then the first exercise device 104-1 may download the exercise program from the remote server 106. A second user may desire to perform the same exercise program on the second exercise device 104-2. Conventionally, if the selected exercise program is not stored locally on the second exercise device 104-2, then the second exercise device 104-2 may download the exercise program from the remote server 106. This multiple downloading of the exercise program may increase the total amount of data downloaded from the remote server 106, resulting in increased server fees.

In accordance with embodiments of the present disclosure, when a user requests to perform an exercise program on the first exercise device 104-1, if the exercise program is not stored locally, then the first exercise device 104-1 may search for the exercise program on the local network 102 before downloading the exercise program from the remote server 106. If another exercise device 104 has the exercise program stored locally, then the other exercise device 104 may transmit the exercise program to the first exercise device 104-1 over the local network 102. In this manner, the first exercise device 104-1 may receive the exercise program from a source other than the remote server 106.

In accordance with embodiments of the present disclosure, transmitting exercise programs across the local network 102 may reduce the amount of data transferred from the remote server 106, thereby reducing server data fees. Furthermore, some gyms may have limited bandwidth to connect to the Internet. Thus, downloading exercise programs from the remote server 106 may take a while. This may delay the start of the user's workout, making the user wait before exercise and decreasing the exercise experience. Transferring exercising programs across the local network 102 may increase transfer speed, thereby decreasing the user's wait time to begin exercising.

In some embodiments, each exercise device 104 may have an active mode and a standby mode. The standby mode may be a low-power mode in which the exercise device 104 uses less than 1 W of power. In some embodiments, transferring information (such as exercise programs) across the local exercise device network 100 may utilize less than 1 W of power. Thus, networked exercise devices 104 may transfer information without coming out of standby mode, or while remaining in a low-power mode. In some embodiments, an exercise device 104 may remain in low power mode to respond to queries regarding which exercise programs are stored in its local memory. If the exercise device 104 receives a request to transfer a particular exercise device, the exercise device 104 may be “woken up” out of low power mode with a ping or other request to transfer the requested exercise program. This may allow more exercise devices 104 to remain on standby, thereby reducing power requirements and reducing the risk of an electrical fire or other electrical complication.

FIG. 2 is a representation of an exercise device network 200, according to at least one embodiment of the present disclosure. The exercise device network 200 includes a local network 202 having two or more interconnected exercise devices 204. The local network 202 shown further includes a local server 210. The local server 210 may be in communication with one or more of the exercise devices 204. In some embodiments, the local server 210 may be in communication with each exercise device 204.

The local network 202 may be in communication with a remote server 206 over the Internet. The remote server 206 may include a database 208 of exercise programs. In some embodiments, the local server 210 may be in communication with the remote server 206. The remote server 206 may transmit one or more of the exercise programs to the local server 210.

In some embodiments, when a user requests an exercise program to use on an exercise device 204, if the exercise program is not stored on the exercise device 204, the exercise device 204 may send a request to the local server 210 for the exercise program. In some embodiments, the local server 210 may then send the exercise program to the user's exercise device 204.

In some embodiments, each of the exercise devices 204 may be in communication with the local server 210. In this manner, the exercise devices 204 may send the request for the exercise program to the local server 210, and the local server 210 may manage the transfer of the exercise program to the user's exercise device 204. In some embodiments, the local server 210 may store the exercise program on memory included as part of the hardware of the local server 210. When the user requests the exercise program, the local server 210 may transfer the exercise program from the memory on the local server 210 to the user's exercise device 204.

In some embodiments, the local server 210 may have a database of which exercise programs are stored on each exercise device 204. When the user requests the exercise program, the local server 210 may identify which exercise device 204 has a copy of the exercise program stored on the local exercise device memory. In some embodiments, the local server 210 may provide an instruction to the exercise device storing the exercise program (e.g., the storage exercise device) to transmit the exercise program to the user's exercise device 204. In some embodiments, the local server 210 may provide an instruction to the user's exercise device to request the exercise program directly from the storage exercise device. In some embodiments, the storage exercise device may transmit the exercise program to the local server 210 and the local server 210 may transmit the exercise program to the user's exercise device 204.

In some embodiments, each exercise program on the local network 202 may be stored on the local server 210. The local server 210 may have a large amount of storage and may be able to store a large number of exercise programs. Whenever a new exercise program becomes available, the local server 210 may receive the new exercise program from the remote server 206 at a convenient time. For example, the local server 210 may be a computing device owned by a commercial gym. The commercial gym may have limited Internet bandwidth when the gym is open. However, after closing, Internet bandwidth may become available, and the local server 210 may request and/or receive any new or requested exercise programs from the remote server 206.

In some embodiments, the local server 210 may be representative of a home gym. For example, the local server 210 may be a personal computing device that is connected or connectable to the exercise devices 204. The personal computing device may provide an interface between the exercise devices 204 and the remote server 206. For example, a user may schedule a workout on one or more of the exercise devices 204 using the personal computing device. In some embodiments, the exercise devices 204 may not be connected to the Internet, and the personal computing device may relay an exercise program between the remote server 206 and the exercise device 204. In some embodiments, the personal computing device may download one or more exercise programs and control elements of the exercise device 204 during implementation of the downloaded exercise program. In some embodiments, the personal computing device may be a mobile phone, such as a smartphone. The display of the personal computing device may be used as a display for the exercise device 204.

In some embodiments, the local server 210 may be encrypted. Encrypting the local server 210 may help to improve the users' data privacy. In some embodiments, the local server 210 (or any device or person on the local network 202) may not have the decryption key. Therefore, even if there is an unauthorized access to the local server 210, the information stored on the local server 210 may not be accessed.

In some embodiments, the exercise devices 204 may only be in communication with the local server 210, and may not be connected to the Internet. This may help to prevent the number of requests to the remote server 206 and reduce the amount of data downloaded from the remote server 206, thereby reducing data fees at the remote server. In some embodiments, the exercise devices 204 may connected to the remote server 206 over the Internet. When the user requests the exercise program, the local server 210 may determine whether the exercise program is stored on the local network 202. If the exercise program is stored on the local network 202, then the local server 210 may manage transmission of the exercise program to the exercise device 204. If the exercise program is not stored on the local network 202, then the local server 210 may download the exercise program from the remote server 206 and then transfer the exercise program to the user's exercise device 204. In some embodiments, the local server 210 may instruct the user's exercise device 204 to download the exercise program from the remote server 206. In some embodiments, the local server 210 may instruct the remote server 206 to push the exercise program to the user's exercise device 204.

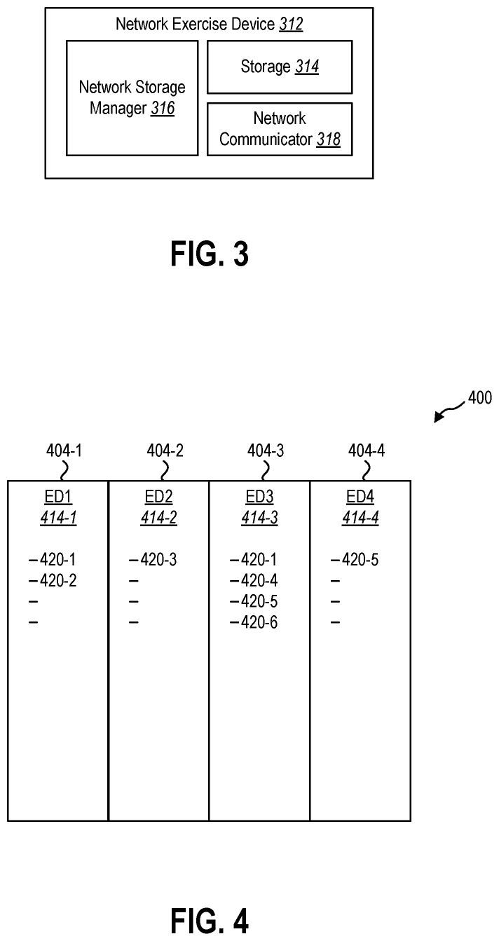

FIG. 3 is a representation of a network exercise device 312 that is part of a local network, such as the local network 102 of FIG. 1, according to at least one embodiment of the present disclosure. The network exercise device 312 includes a local storage 314. The local storage 314 may include one or more exercise programs. A user may use the network exercise device 312 and request to perform an exercise program. In some embodiments, the local storage 314 may include a list of available exercise programs. In some embodiments, the available exercise programs may include a listing of all of the exercise programs stored on the local storage 314. In some embodiments, the available exercise programs may include a listing of every exercise program available to the user, either locally stored, stored on an exercise device in the local network, and/or stored on a remote server (such as the remote server 106 of FIG. 1). In some embodiments, the list of available programs may include an indication of the storage location of each of the exercise programs.

In some embodiments, the user may select an exercise program that is not stored in the local storage 314. In some embodiments, a network storage manager 316 may determine where the selected exercise program is stored. For example, the network storage manager 316 may utilize a network communicator 318 to communicate with other network exercise devices to determine if the exercise program is located on the local network. If the exercise program is located on a network exercise device connected to the local network, the network communicator 318 may receive the exercise program from the connected network exercise device. If the exercise program is not located on the local network, then the network storage manager 316 may send a request for the exercise device from the remote server, and the network communicator 318 may receive the exercise device from the remote server.

FIG. 4 is a representation of the local storages (collectively 414) of an exercise device network 400, according to at least one embodiment of the present disclosure. The exercise device network 400 may include a plurality of exercise devices (collectively 404), each having a local storage (collectively 414). The local storages 414 may store one or more exercise programs. In some embodiments, the local storages 414 may store a set of exercise programs. For example, a first local storage 414-1 may include a first set of exercise programs 420 and a second local storage 414-2 may include a second set of exercise programs 420. As may be seen in FIG. 4, a given set of exercise programs 420 on an exercise device 404 may include at least one exercise program not included on the local storage 414 of another exercise device. For example, the first set of exercise programs stored on the first local storage 414-1 includes the first exercise program 420-1 and the second exercise program 420-2. The second set of exercise programs stored on the second local storage 414-2 includes the third exercise program 420-3. The first set of exercise programs does not include the third exercise program 420-3, and the second set of exercise programs does not include the first exercise program 420-1 and the second exercise program 420-2.

Each of the exercise devices 404 may be in direct communication with each other over a local network. In some embodiments, when the exercise devices 404 are in direct communication with each other over the local network, the exercise devices 404 may communicate with each other without sending data over the Internet, or without a remote server negotiating the communication, or without a server negotiating the communication.

The local storages 414 may have a storage capacity. While the storage capacity is shown in FIG. 4 as a number of “slots,” or number of available storage locations used to store exercise programs, it should be understood that the local storages 414 have a storage capacity based on the available amount of memory, which may be expressed as an amount of MB, GB, TB, or more. For simplicity of illustration, it is assumed that each exercise program is the same size, and therefore, based on the size of the exercise program and the available memory, each exercise device 404 may have a number of slots for storing exercise programs. However, it should be understood that the principles of the present disclosure may be applied to analyzing the available memory of the exercise devices 404 and the size of the exercise programs (which may be variable between different exercise programs) to determine if there is room in the exercise device 404 for the exercise program.

In accordance with embodiments of the present disclosure, if the selected exercise program is not located on local storage, the exercise program may be downloaded to the first exercise device. However, if the first exercise device has no available storage, then one or more stored exercise programs may be deleted or removed from the local storage of the exercise device.

In accordance with embodiments of the present disclosure, when a user wishes to perform a first exercise program 420-1 on a first exercise device 404-1, the user may select the first exercise program 420-1 and the first exercise device 404-1 may implement the first exercise program 420-1 stored locally on the first local storage 414-1. However, if the user wishes to perform the third exercise program 420-3, the first exercise device 404-1 may receive the third exercise program 420-3 stored in the second local storage 414-2 of the second exercise device 404-2. When the first exercise device 404-1 may receive and store the third exercise program 420-3 on the first local storage 414-1. After the third exercise program 420-3 is stored on the first local storage 414-1, the first exercise device 404-1 may implement the third exercise program 420-3 recently stored in the first local storage. Thus, as may be seen, the user may access any of the exercise programs 420 stored on any of the exercise devices 404, including the first exercise program 420-1, the second exercise program 420-2, the third exercise program 420-3, the fourth exercise program 420-4, the fifth exercise program 420-5, the sixth exercise program 420-6, and combinations thereof.

In some embodiments, an exercise program 420 may be stored on a type of exercise device 404 for which the exercise program 420 is designed. For example, the exercise program 420 may be a treadmill exercise program, and the exercise program 420 may be stored on a treadmill. In some embodiments, the exercise program may be stored on a type of exercise device for which the exercise program is not designed. For example, the exercise program 420 may be a rower exercise program, and the exercise program 420 may be stored on a stationary exercise cycle. By storing exercise programs on exercise devices 404 for which the exercise program 420 is not designed, the storage of the entire exercise device network 400 may be optimized.

In some embodiments, the exercise program 420 may be transferred to the requesting exercise device 404 from a single seed exercise device 404. In some embodiments, the exercise program 420 may be stored on multiple exercise devices 404, and the exercise program 420 may be transferred to the requesting exercise device 404 from multiple seed exercise devices 404. Multiple seed exercise devices may increase the rate of transfer of the exercise program, allowing it to be downloaded to the requesting exercise device 404 quickly and efficiently. For example, to transfer the exercise program 420, the exercise program 420 may be split into multiple different portions or packets. Transferring the exercise program from multiple seed devices may transfer one portion (e.g., a first packet) from a first seed exercise device 404 and a second portion (e.g., a second packet) from a second exercise device 404. In this manner, the exercise program 420 may be transferred faster and more reliably by transmitting the exercise device from multiple seed exercise devices 404.

In some embodiments, the requested exercise program 420 may be completely transferred between exercise devices 404 over the exercise device network 400. Put another way, the requested exercise program 420 may be transferred between exercise devices 404 over the exercise device network without transferring any data over the Internet. In some embodiments, a program percentage (e.g., amount transferred divided by total file size) of the exercise program 420 may be transferred over the Internet. In some embodiments, the program percentage may be in a range having an upper value, a lower value, or upper and lower values including any of 10%, 5%, 2.5%, 1%, 0.5%, 0.25%, 0.1%, 0.01%, or any value therebetween. For example, the program percentage may be greater than 0.01%. In another example, the program percentage may be less than 10%. In yet other examples, the program percentage may be any value in a range between 0.01% and 10%. In some embodiments, it may be critical that the program percentage is less than 1% to reduce bandwidth requirements and server access fees.

In some embodiments, an internet download size is the amount of information transferred over the Internet to download the exercise program. In some embodiments, the internet download size may be in a range having an upper value, a lower value, or upper and lower values including any of 1 kilobyte (kb), 10 kb, 50 kb, 100 kb, 500 kb, 1 Megabyte (Mb), 5 Mb, 10 Mb, 50 Mb, 100 Mb, or any value therebetween. For example, the internet download size may be greater than 1 kb. In another example, the internet download size may be less than 100 Mb. In yet other examples, the internet download size may be any value in a range between 1 kB and 100 Mb. In some embodiments, it may be critical that the internet download size is less than 1 Mb to reduce bandwidth requirements and server access fees.

In some embodiments, the local storage 414 of an exercise device 404 may be full. For example, the third exercise device 404-3 may have a third local storage 414-3 that is full, meaning that there is not enough remaining memory to download an entire exercise program 420. For example, while there may be remaining storage on the third local storage 414-3, there may not be sufficient remaining memory to download the entire selected exercise program 420. In some embodiments, there may be sufficient remaining memory to download other exercise programs 420, but not the selected memory program.

When the user selects an exercise program 420 to be used on the third exercise device 404-3, such as the second exercise program 420-2, the exercise device 404 may determine whether there is sufficient memory in the third local storage 414-3 to download the second exercise program 420-2. If there is not sufficient memory in the third local storage 414-3, then, as discussed in further detail herein, one or more of the downloaded exercise programs 420 stored on the third local storage 414-3 (e.g., the first exercise program 420-1, the fourth exercise program 420-4, the fifth exercise program 420-5, or the sixth exercise program 420-6) may be deleted from the third local storage 414-3. This may free up storage capacity on the third local storage 414-3 and allow the second exercise program 420-2 to be downloaded to the third local storage 414-3.

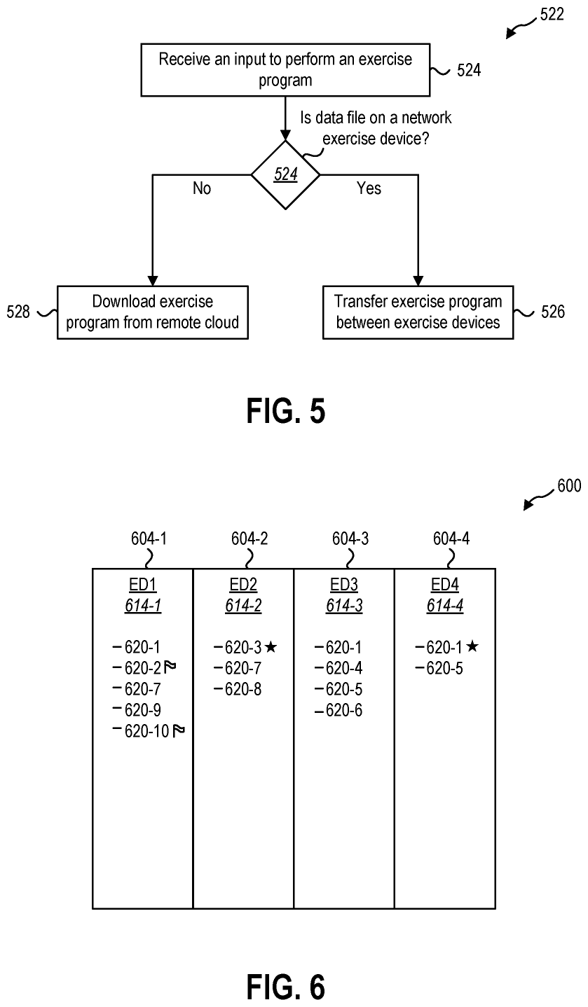

FIG. 5 is a flowchart of a method 522 for implementing an exercise program, according to at least one embodiment of the present disclosure. The method 522 may be implemented on the exercise device network 100 of FIG. 1. Put another way, exercise device network 100 may implement the method 522 of FIG. 5. The method 522 may include receiving an input to perform an exercise program at 524. In some embodiments, the input may be received at the exercise device to be used. In some embodiments, the input may be received from a remote device. For example, the input may be received at an exercise manager, or a local server connected to each of the exercise devices. For example, the exercise devices on the exercise network may be connected to a mobile device over the Internet. The user may plan to perform an exercise on a specific exercise device. The user may select the exercise program to be performed and the exercise device on which to perform the exercise program. In some embodiments, the user may “reserve” the exercise device for a specific period of time to perform the exercise program.

After the input is received, the exercise device may determine whether the data file of the exercise program is located in the exercise device network at 524. For example, the exercise device (or local server, or remote server), may search the local storage of each exercise device for whether the exercise network includes the data file of the exercise program. In some embodiments, determining whether the exercise program data file is located on the exercise network may include querying the exercise network and/or each exercise device on the exercise network for the exercise program. Querying the exercise network may help to determine which, if any, of the exercise devices on the exercise network has stored the exercise program on local storage. If one of the exercise devices on the exercise network has stored the exercise program on local storage, the exercise device may provide a positive response to the query. If an exercise device does not have the exercise program stored, it may remain silent or provide a negative response.

In some embodiments, determining whether the data file is located on the exercise network may include checking an exercise program database stored on the local network. The exercise program database may include a listing of which exercise programs are stored on which exercise devices on the local network. The requesting exercise device may check the exercise program database to determine which exercise device(s) have stored the exercise program. The requesting exercise device may then provide an individual request for the exercise program to the exercise device(s) which has the exercise program stored. In some embodiments, a local server may have the exercise program database stored and determine whether the data file is located on the exercise network may include querying the local server. The local server may provide a response regarding which exercise device(s) (if any) are storing the exercise program.

If the data file is located on the exercise network, then the exercise program may be transferred between exercise devices on the exercise device network to the user's exercise device at 526. If the data file is not located on the exercise network, then the exercise program may be downloaded from the cloud at 528. This may help to prevent multiple downloads of the exercise program to devices on the exercise device network, thereby reducing data fees on the cloud.

FIG. 6 is a representation of the local storages (collective 614) of an exercise device network 600, according to at least one embodiment of the present disclosure. The exercise device network 600 may include a plurality of exercise devices (collectively 604), each having a local storage (collectively 614). The exercise device network 600 shown includes a first exercise device 604-1 having a first local storage 614-1, a second exercise device 604-2 having a second local storage 614-2, a third exercise device 604-3 having a third local storage 614-3, and a fourth exercise device 604-4 having a fourth local storage 614-4. The local storages 614 may store one or more exercise programs (collectively 620). The exercise programs 620 may include a first exercise program 620-1, a second exercise program 620-2, a third exercise program 620-3, a fourth exercise program 620-4, a fifth exercise program 620-5, a sixth exercise program 620-6, a seventh exercise program 620-7, an eighth exercise program 620-8, a ninth exercise program 620-9, and a tenth exercise program 620-10. Each of the exercise devices 604 may be in communication with each other.

In accordance with embodiments of the present disclosure, when a user wishes to perform a first exercise program 620-1 on a first exercise device 604-1, the user may select the first exercise program 620-1 and the first exercise device 604-1 may implement the first exercise program 620-1 stored locally on the first local storage 614-1. However, if the user wishes to perform the third exercise program 620-3, the third exercise program 620-3 may need to be transferred to the first local storage 614-1.

In the embodiment shown, the first local storage 614-1 may be full (e.g., the first local storage 614-1 may not have sufficient remaining storage to download another exercise program 620). Thus, to install the third exercise program 620-3 on the first exercise device 604-1, one or more of the exercise programs 620 stored on the first local storage 614-1 may need to be deleted. In some embodiments, one or more of the exercise programs 620 on the first local storage 614-1 may be deleted. In some embodiments, deleting an exercise program 620 may include wiping the exercise program 620 from the local storage 614. In some embodiments, deleting the exercise program 620 may include marking the memory allocated to the exercise program for over-writing or replacement.

In some embodiments, deleting the exercise program 620 may include transferring the exercise program 620 to the local storage 614 of a different exercise device 604. For example, the exercise program 620 may not be stored on any other local storage 614 of any exercise device. To maintain the exercise program 620 on the exercise device network 600, the exercise program 620 may be transferred to another exercise device before being deleted from the local storage 614.

In some embodiments, one or more exercise programs 620 may be flagged for deletion. For example, the user may flag one or more exercise programs 620 for use. For example, the user may identify or select one or more exercise programs 620 to be deleted. The user may identify or select the one or more exercise programs 620 to be deleted based on any reasoning. For example, the user may desire to perform a new exercise program every time he or she exercises, the user may feel that he or she has performed a particular exercise program enough times, the user may arbitrarily identify an exercise program for deletion, any other reasoning, and combinations thereof. In some embodiments, the user may flag an exercise program 620 for deletion based on a prompt from the exercise device 604. For example, when the user selects an exercise program 620 not located on the exercise device 604, the exercise device 604 (or other input device) may prompt the user to select an exercise program 620 for deletion.

In some embodiments, the exercise device 604 may identify or flag one or more exercise programs 620 for deletion or replacement. In some embodiments, the exercise device network 600 may flag an exercise program 620 based on network-wide metrics. For example, the exercise device 604 may identify low-utilization exercise programs 620 (e.g., the least-used exercise programs 620) for deletion. In some embodiments, the low-utilization exercise program 620 may have the lowest number of uses when a new exercise program 620 is selected for download. In some embodiments, the least-used exercise program 620 may have the longest time between last use when a new exercise program 620 is selected for download. In some embodiments, the low-utilization exercise program 620 may have the lowest user rating of all the exercise programs 620 on the exercise device 604. In some examples, the exercise device 604 may flag for deletion the exercise program 620 that has been on the exercise device 604 the longest. In some examples, the exercise device 604 may flag for deletion the exercise program 620 that has the largest data file size. In some examples, the exercise device 604 may flag for deletion the exercise program that has the smallest data file size for which a deletion may provide room on the local storage 614 for the selected exercise program.

In some embodiments, the exercise device network 600 may identify or flag one or more exercise programs 620 for deletion or replacement. For example, the exercise device network 600 may identify or flag an exercise program 620 for deletion if there are one or more copies of the exercise program 620 on other exercise devices 604 within the exercise device network 600. In some examples, the exercise device network may identify or flag the least-used exercise programs 620 across the entire exercise device network 600. For example, despite the first exercise device 604-1 having run the second exercise program 620-2 multiple times, the total usage of the second exercise program 620-2 on the other exercise devices 604 of the exercise device network 600 may be low, resulting in the second exercise program 620-1 being flagged for deletion.

In some embodiments, the exercise device network 600 may receive instructions from the cloud to identify or flag one or more exercise programs 620 for deletion. For example, a specific exercise program 620 may have discontinued cloud support, may have low overall usage across multiple exercise device networks, and may be flagged for replacement by more highly rated or newer exercise programs 620.

In some embodiments, when the user selects an exercise program 620 to download onto an exercise device 604, the exercise device 604 may delete or replace the flagged exercise program with the selected exercise program. For example, in the embodiment shown in FIG. 6, the user may desire to perform the fourth exercise program 620-4, which is stored on the third exercise device 604-3, on the first exercise device 604-1. Upon making the selection, the first exercise device 604-1 may delete one or more flagged exercise programs 620 (e.g., the second exercise program 620-2 and/or the tenth exercise program 620-10) to make room for the fourth exercise program 620-4.

In some embodiments, one or more exercise programs 620 may be starred or otherwise identified (e.g., flagged, marked) to be retained (e.g., be prevented from deletion). When a user selects an exercise program 620 that is not located on an exercise device 604, the exercise device 604 may not delete the starred exercise program 620. In some embodiments, the user may star an exercise program 620. For example, the user may star an exercise program 620 because he or she particularly likes the exercise program 620, the trainer, the exercise device 604, and combinations thereof.

In some embodiments, the exercise device 604 may star an exercise program 620. For example, the exercise device 604 may star an exercise program 620 that has the most uses, the most recent uses, the highest user reviews, and combinations thereof. In some examples, the exercise device 604 may star exercise programs 620 that have been recently downloaded. Put another way, the exercise device 604 may star exercise programs 620 that have been downloaded within a period of time, such as an hour, a day, a week, a month, and so forth.

In some embodiments, the exercise device network 600 may star one or more exercise programs 620. In some embodiments, the exercise device network 600 may star an exercise program 620 based on network-wide metrics. For example, the exercise device network 600 may star an exercise program 620 based on network-wide high usage or high user rating, based on how recently it was downloaded to the network for any other reason, and combinations thereof. In some embodiments, the cloud may instruct the exercise device network 600 to star an exercise program 620. For example, the cloud may instruct the exercise device network 600 to star exercise programs 620 that are popular across multiple networks, that are newly released, promotional exercise programs 620, seasonal exercise programs 620, for any other reason, and combinations thereof.

In the embodiment shown, the fourth exercise device 604-4 includes the first exercise program 620-1 and the fifth exercise program 620-5. The first exercise program 620-1 is shown as starred. Thus, if a user wishes to implement an exercise program 620 on the fourth exercise device 604-4, the fifth exercise program 620-5 may be deleted and the new exercise program 620 downloaded.

When planning a workout, the user may be presented with a list of available exercise programs 620. The list of available exercise programs 620 may include indications regarding on which exercise device 604 a particular exercise program 620 is located. This may provide an indication to the user regarding how fast a particular exercise program 620 may be loaded onto the exercise device 604. For example, if a user wishes to perform the third exercise program 620-3 on the first exercise device 604-1, the list of available exercise programs 620 may indicate that the third exercise program 620-3 is located on an exercise device in the exercise network. This may indicate to the user that the third exercise program 620-3 may be transferred to the first exercise device 604-1 (e.g., from the second exercise device 604-2) relatively quickly. However, if the user wishes to perform an exercise program not located on any device within the exercise device network 600, the user may infer that downloading the exercise program from the cloud to the first exercise device 604-1 may take longer.

In some embodiments, the cloud server may push one or more updated exercise programs 620 to one or more exercise devices 604 on the exercise device network 600. For example, a new exercise program 620 may be released. The cloud server may push the newly released exercise program 620 to the exercise device network 600. In this manner, the cloud server may control when a particular exercise device network 600 may receive the new exercise program 620. This may help to balance the data traffic load on the cloud server.

In some embodiments, an exercise device network 600 may have limited access to the Internet. For example, the exercise device network 600 may have limited Internet bandwidth, data caps, or other limitations on the Internet access. In some embodiments, a new exercise program 620 may be downloaded to an exercise device 604 during low Internet access or usage. For example, access to the Internet is usually lower at night. To preserve daytime bandwidth, the cloud server may push a new exercise program 620 to the exercise device network 600 at night. In some embodiments, the exercise device network 600 may request or download the new exercise program 620 from the cloud server at night.

In some embodiments, the user may schedule to perform an exercise program 620 for a future time. The future time may be minutes, hours, days, or weeks in the future of when the user makes a selection of an exercise program 620. If the user schedules to perform the exercise program in the future, the exercise device 604 and/or the exercise network may schedule the download of the exercise program 620 for a time that optimizes network bandwidth. This may free up Internet bandwidth for the exercise device network 600, thereby allowing increased user access (e.g., for music/video streaming) and/or increased gym access (e.g., for exercise information up/download).

As may be seen, different exercise devices 604 may have different internal storage capacities. In some embodiments, a managing server of the exercise device network 600 may manage the storage of the different exercise devices 604. The managing server may flag or star different exercise programs 620, manage downloading of new exercise programs, perform any other management task, and combinations thereof. In some embodiments, an exercise device network 600 may include a local storage device, such as a computer, external hard drive, local server, and so forth. The local storage device may maintain a library of exercise programs 620. When a user wishes to perform an exercise program 620 not stored on a particular exercise device 604, the local storage device may transfer the selected exercise program 620 to the exercise device 604 over the local area network for the exercise device network 600.

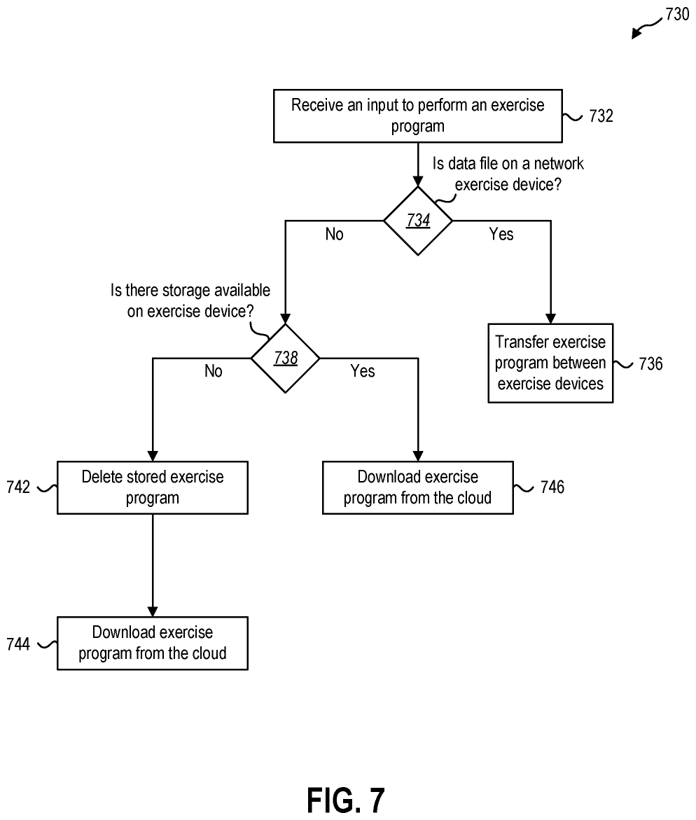

FIG. 7 is a flowchart of a method 730 for implementing an exercise program, according to at least one embodiment of the present disclosure. The method 730 may be implemented on the exercise device network 100 of FIG. 1. Put another way, exercise device network 100 may implement the method 730 of FIG. 7. The method 730 may include receiving an input to perform an exercise program at 732. After the input is received, the exercise device may determine whether the data file of the exercise program is located on an exercise device of the exercise device network at 734. If the data file is located on an exercise device of the exercise device network, the exercise program may be transferred between exercise devices over the exercise device network at 736.

If the data file is not located on the exercise device, the exercise device may determine whether there is storage available on the local storage of the exercise device at 738. If storage is available on the local storage, then the exercise program may be downloaded to the exercise device from the cloud server at 746. If storage is not available on the local storage, then one or more stored exercise programs stored on the exercise device may be deleted from the local storage at 742. The selected exercise program may then be downloaded from the cloud at 744.

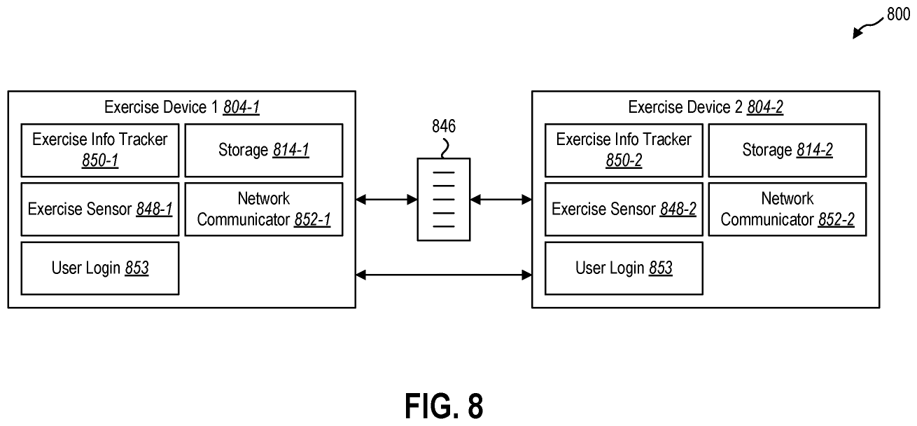

FIG. 8 is a representation of an exercise device network 800 having exercise devices (collectively 804) on which the user may implement a single exercise program, according to at least one embodiment of the present disclosure. In some embodiments, a user may wish to perform a single workout on multiple exercise devices 804. For example, an exercise program may include a first portion that is to be performed on a first exercise device 804-1, such as a treadmill, and a second portion that is to be performed on a second exercise device 804-2. The user may desire to track exercise information while performing the exercise. Exercise information may include heartrate, time, calories burned, speed, distance, steps, cadence, any other exercise information, and combinations thereof.

In some embodiments, the user may wish to collect information that is continuous across both the first portion and the second portion of the exercise program. For example, the user may wish to have an average heartrate, total time, total calories burned, average speed, total distance, total steps, average cadence, any other total or average exercise information, and combinations thereof, that is cumulative or averaged across the entire workout.

Because the workout includes portions on both the first exercise device 804-1 and the second exercise device 804-2, each exercise device 804 may collect exercise information using one or more exercise sensors 848. The exercise sensors 848 may include any exercise sensor, including heart rate sensors, speed sensors, pressure sensors, weight sensors, any other sensor, and combinations thereof. In some embodiments, the exercise devices 804 may track the collected exercise information using one or more exercise information tracker 850. In some embodiments, each exercise device 804 may store the exercise information on local storage 814. For example, the first exercise device 804-1 may collect exercise information using a first exercise sensor 848-1 and track the exercise information on a first local storage 814-1 using a first exercise information tracker 850-1. In some examples, the second exercise device 804-2 may collect exercise information using a second exercise sensor 848-2 and track the exercise information on a second local storage 814-2 using a second exercise information tracker 850-2.

In some embodiments, the exercise devices 804 may track cumulative and/or average exercise information on a combined exercise information file 846. The exercise devices 804 may be connected to each other over the exercise device network 800. In some embodiments, the first exercise device 804-1 may be connected to the second exercise device 804-2 over the exercise device network 800 using a first network communicator 852-1. The second exercise device 804-2 may be connected to the first exercise device 804-1 over the exercise device network 800 using a second network communicator 852-2. In this manner, each exercise device 804 may modify the exercise information file 846 over the exercise device network 800. In some embodiments, each exercise device 804 may modify the exercise information file 846 without transferring any information over the Internet. For example, all the collected information may be transferred over the exercise device network 800.

In some embodiments, the combined exercise information file 846 may be stored remote from the exercise devices 804. For example, the combined exercise information file 846 may be stored on a local server or computer. When the user performs a first portion of the exercise program on the first exercise device 804-1, the first exercise device 804-1 may collect the exercise information with the first exercise sensor 848-1 and record the exercise information on the exercise information file 846 using the first exercise information tracker 850-1 via the first network communicator 852-1. When the user performs a second portion of the exercise program on the second exercise device 804-2, the second exercise device 804-2 may collect the exercise information with the second exercise sensor 848-2 and record the exercise information on the exercise information file 846 using the second exercise information tracker 850-2 via the second network communicator 852-2.

In some embodiments, the combined exercise information file 846 may be stored on the first local storage 814-1. The first exercise device 804-1 may record the exercise information on the exercise information file 846 directly on the first local storage 814-1 using the first exercise information tracker 850-1. The second exercise device 804-2 may record the exercise information on the exercise information file 846 on the first local storage 814-1 using the second exercise information tracker 850-2 and the second network communicator 852-2.

In some embodiments, the combined exercise information file 846 may be stored on the second local storage 814-2. In some embodiments, the second exercise device 804-2 may record the exercise information on the exercise information file 846 directly on the second local storage 814-2 using the second exercise information tracker 850-2. The first exercise device 804-1 may record the exercise information on the exercise information file 846 on the second local storage 814-2 using the first exercise information tracker 850-1 and the first network communicator 852-1. In some embodiments, the combined exercise information file 846 may be stored on each exercise device 804. Updates to the exercise information file 846 may be transmitted to each exercise device 804 using the network communicators.

In some embodiments, the combined exercise information file 846 may be stored on the first exercise device 804-1 during the first portion of the exercise program. After the user finishes the first portion of the exercise program, the combined exercise information file 846 may be transmitted to the second exercise device 804-2 and the combined exercise information file 846 removed from the first exercise device 804-1. This may help to reduce the number of locations that the user's data is stored, thereby improving the user's information privacy and/or sense of information privacy.

Conventionally, a combined exercise information file 846 may be stored on the cloud server. Thus, every update to the combined exercise information file 846 may be uploaded to the cloud and then downloaded to the exercise device being used. This may increase the amount of network traffic over the Internet connection to remote cloud. As discussed herein, the Internet bandwidth may be limited. This may result in reduced transfer of the exercise information between exercise devices 804. This may further result in exercise delays as the user waits for the exercise information to be transferred to the next exercise device 804.

As discussed herein, updating the combined exercise information file 846 over the exercise device network 800 may reduce or prevent the amount of information transmitted to the cloud server over the Internet. Because the exercise devices 804 are connected over the exercise device network 800, the transfer of exercise information may be fast, or instantaneous. This may help to reduce the delay between portions of the exercise program. This may improve the user experience by allowing the user to maintain his or her heartrate at a desired level when transitioning between exercise devices.

In some embodiments, updating and storing the combined exercise information file 846 on the local exercise device network 800 may allow the exercise device network to upload the combined exercise information file 846 to a remote server anytime. For example, the exercise device network 800 may upload the combined exercise information file 846 during low-usage times of the exercise device network 800's Internet connection. This may help to make Internet bandwidth available for other uses, such as media access, downloading of new exercise programs, and so forth.

When the user begins performing an exercise on an exercise device 804, he or she may log in to the exercise device 804 using a user login 853. Using the user login 853, the user may log on to each exercise device 804 in any manner, including a user ID and password, an RFID identifier, near-field communication identifier, biometric identifier (such as fingerprint, EKG pattern, facial recognition, voice recognition, etc.), any other login information, and combinations thereof.

While embodiments of the present disclosure have discussed an exercise device network 800 tracking exercise information for a single user's exercise program, it should be understood that the exercise device network may track multiple user's exercise information on multiple shared exercise information files 846. For example, a first user may perform a portion of a first exercise program on a first exercise device 804-1, and the first exercise device 804-1 may track the associated exercise information on a first shared exercise information file. When the portion is complete, the first user may move to a second exercise device 804-2, which may continue to track the user's exercise information using the first shared exercise information file. When the first user finishes using the first exercise device 804-1, a second user may begin using the first exercise device 804-1. The first exercise device 804-1 may track the second user's associated exercise information on a second shared exercise information file. Thus, the exercise device network 800 may track multiple shared exercise information files simultaneously. For example, the first exercise device 804-1 may update the second shared exercise information file while the second exercise device 804-2 is simultaneously updating the first shared exercise information file.

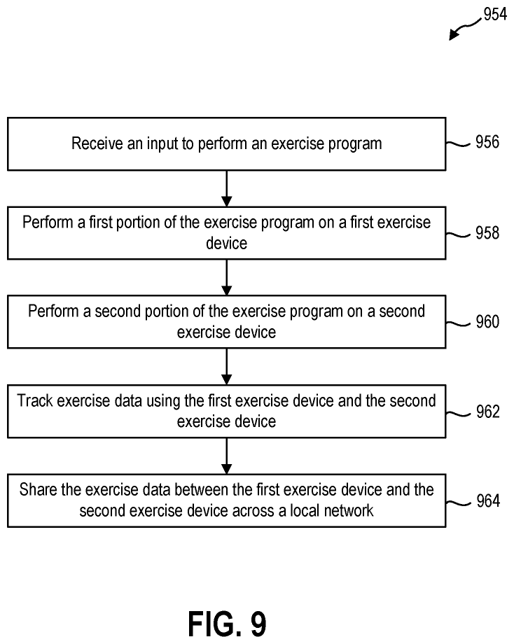

FIG. 9 is a representation of a method 954 for implementing an exercise program, according to at least one embodiment of the present disclosure. The method 954 include receiving an input to perform an exercise program at 956. As discussed herein, the input may be received at any location. For example, a user may provide an input on an interactive display of an exercise device. In some examples, the user may provide the input on a remote computing device. For example, the user may provide the input using an app on a mobile device or other remote computing device.

The exercise program may be performed across multiple exercise devices. For example, the exercise program may include a plurality of portions. A first portion may be performed on a first type of exercise device (such as a rower) and a second portion may be performed on a second type of exercise device (such as an elliptical trainer). In some examples, the first exercise device and the second exercise device may be the same type of exercise device. For example, a user may perform the first portion on the first exercise device (such as a stationary exercise cycle). The user may dismount or otherwise leave the first exercise device after the first portion is over (such as for a rest, a restroom break, stretching, or any other reason). While the user has left the first exercise device, another user may begin using the first exercise device. When it is time to perform the second portion of the exercise program, the user may begin using the second exercise device of the same type as the first exercise device without interrupting his exercise program. By sharing exercise information across exercise devices, the user may experience greater flexibility regarding which exercise devices he or she may use. Furthermore, this may help increase exercise device utilization at a gym, especially during peak usage time, by reducing exercise device downtime.

In some examples, the first portion may be performed on a first type of exercise device, and the second portion may be performed using free-weights (including free weights connected to the exercise device network). The exercise program may include a third portion that is performed on the first type of exercise device. In some embodiments, the first portion and the third portion may be performed on the same exercise device. In some embodiments, the first portion and the third portion may be performed on different exercise devices of the same type. For example, the user may perform the exercise program in a crowded gym. While the user is performing the second portion (e.g., using the free weights), a different user may begin using the first exercise device. When the user finishes the second portion, he may continue the exercise on a second exercise device of the same type as the first exercise device.

The user may perform the first portion of the exercise program on a first exercise device at 958. While performing the first portion of the exercise program, the first exercise device may track exercise information about the user and/or the exercise program on a shared exercise information file. The user may then perform the second portion of the exercise program on a second exercise device at 960. The second exercise device may track the exercise information related to the second portion on the shared exercise information file at 962. The shared exercise information file may be shared between the first exercise device and the second exercise device at 964.

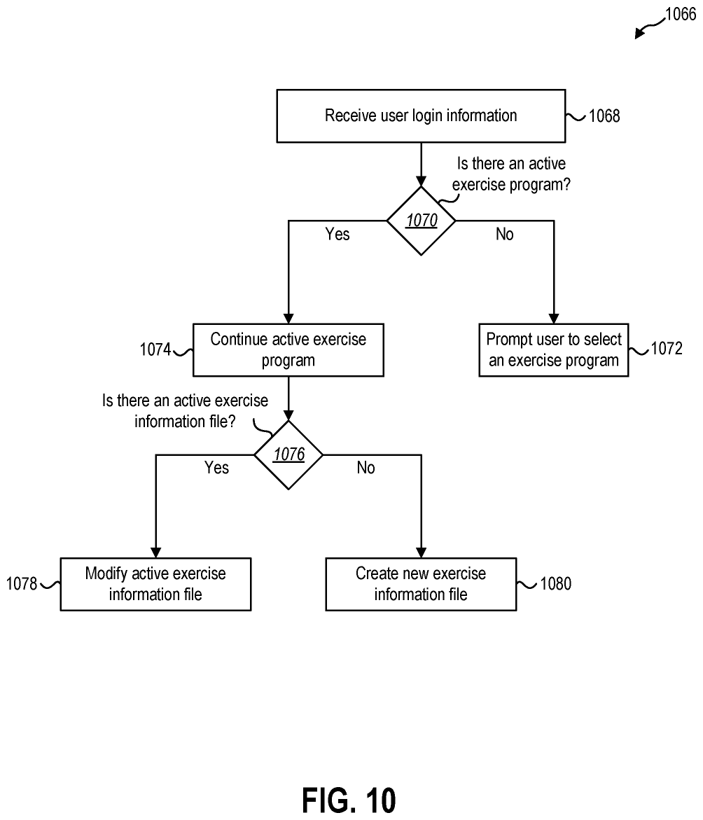

FIG. 10 is a flowchart of a method 1066 for implementing an exercise program on an exercise device network, according to at least one embodiment of the present disclosure. When the user begins performing an exercise on an exercise device, he or she may log in to the exercise device causing the exercise device to receive user login information at 1068. The user login information may be any type of user login information, including a user ID and password, an RFID identifier, near-field communication identifier, biometric identifier (such as fingerprint, EKG pattern, facial recognition, voice recognition, etc.), any other login information, and combinations thereof. After the user logs in, the exercise device may determine whether there is an active exercise program associated with the user at 1070. An active exercise program may be an exercise program that the user has selected and performed at least a portion of on another exercise device. If the exercise device determines that there is not an active exercise program associated with the user, the exercise device may prompt the user to select an exercise program to be implemented at 1072.

If the exercise device determines that there is an active exercise program associated with the user, the exercise device may continue the active exercise program at 1074. Continuing the active exercise program may include determining which portion(s) of the exercise program have been completed, and continuing the exercise program by implementing the next portion. In some embodiments, if portion of the exercise program is inconsistent with the type of exercise device, then the exercise device may prompt the user to move to the appropriate exercise device.

When continuing the active exercise program, the exercise device may further determine whether there is an active exercise information file associated with the user on the exercise device network at 1076. If there is an active exercise information file associated with the user, then the exercise device may modify the active exercise information file with exercise information relevant to the portion of the exercise program being performed at 1078. Put another way, the exercise device may track exercise information to the active exercise information file for the user. If there is not an active exercise information file associated with the user on the exercise device network, then the exercise device may create an active exercise information file for the user at 1080. The exercise device may then track exercise information to the active exercise information file.

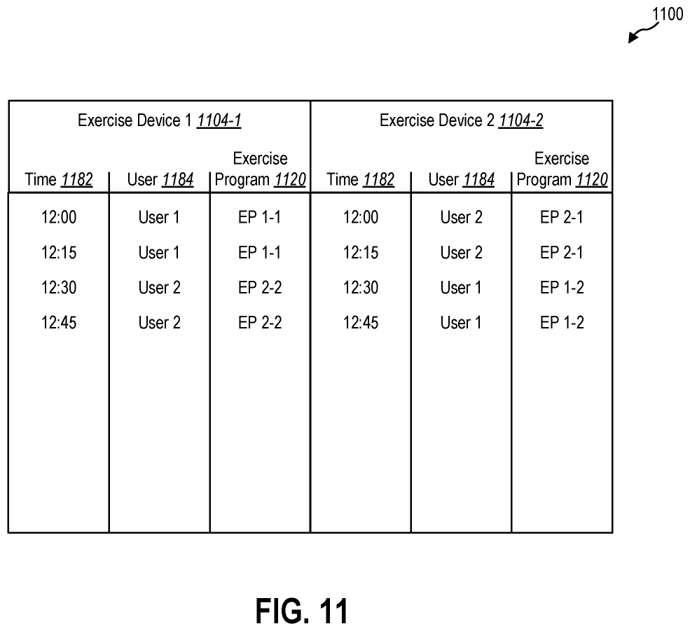

FIG. 11 is a representation of a schedule of an exercise device network 1100, according to at least one embodiment of the present disclosure. On the exercise device network 1100, a user 1184 may schedule an exercise program 1120 to be performed in a particular time slot 1182 of an exercise device (collectively 1104). Reserving a single exercise program 1120 across multiple exercise devices 1104 may help a user to complete an exercise program 1120 across multiple exercise deices 1104 uninterrupted, or without waiting for an exercise device 1104 to become available.

In the embodiment shown, a first user 1184 may reserve the first exercise device 1104-1 from a time 1182 of between 12:00 and 12:30 to perform a first portion of a first exercise program 1120. The first user 1184 may then reserve the second exercise device 1104-2 from a time 1182 of between 12:30 and 1:00 to perform a second portion of the first exercise program 1120. If another user tries to log in to the first exercise device 1104-1 when the first exercise device 1104-1 is reserved by the first user 1184, the first exercise device 1104-1 may not function for the second user. This may help a user to know that he or she has an uninterrupted time available to exercise on the first exercise device 1104-1.

As may be seen, the exercise devices 1104 may be scheduled based on the duration of individual portions of an exercise program 1120. For example, after the user completes the first portion of the first exercise program 1120, the second user may perform a portion of an exercise program on the first exercise device 1104-1. In some embodiments, the second user may perform the portion of the exercise program 1120 while the first user is performing the second portion of the first exercise program 1120. In this manner, the first exercise device 1104-1 may not be scheduled for the entire duration of the exercise program 1120, but rather for the duration of the relevant portion of the exercise program 1120. This may help to increase exercise device utilization, thereby increasing the capacity of the gym.

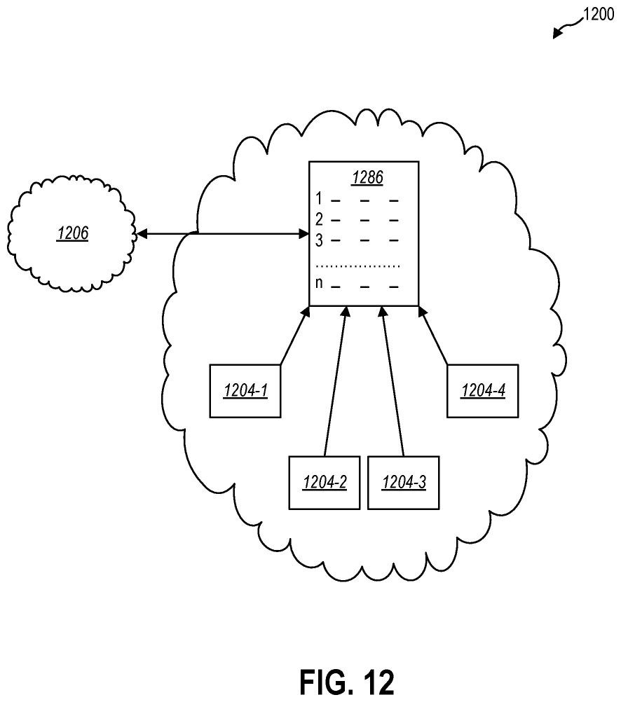

FIG. 12 is a representation of an exercise device network 1200 having a local leaderboard 1286, according to at least one embodiment of the present disclosure. During a group exercise session, multiple users may perform the same exercise program on multiple exercise devices 1204-1, 1204-2, 1204-3, 1204-4 (collectively 1204). Each exercise device 1204 may collect exercise information about their associated user. The collected exercise information may be different based on the different abilities and fitness levels of the different users. In some embodiments, users may wish to compare at least a portion of their exercise information with other users. For example, a user may wish to compare his or her speed, distance, time, power, and other exercise information against other users that have used the same exercise program. In some embodiments, a group of user's exercise information may be presented as a leaderboard, where the leaderboard includes a summary of relevant exercise information for various users, often sorted by speed, distance, power, and so forth.

Conventionally, leaderboard information is collected from a remote server 1206 (e.g., the cloud). Each exercise device 1204 may be connected to the remote server 1206, and may upload the relevant exercise information to the remote server 1206, and the leaderboard 1286 may be created using the exercise information stored on the remote server 1206. In this manner, a user may compare him or herself against remote users, including global users, national users, and local users. In some embodiments, a leaderboard of a particular exercise group may be collected from the remote server 1206. For a large database on the remote server 1206, the time to search, sort, and send all of the leaderboard information may become cumbersome, resulting in delays to the update of the leaderboard 1286.

In accordance with embodiments of the present disclosure, an exercise device network 1200 may prepare a local leaderboard 1286. The exercise information presented on the local leaderboard 1286 may be collected from the networked exercise devices 1204 connected to the exercise device network 1200. Each exercise device 1204 may transmit the exercise information over the exercise device network 1200 to the local leaderboard 1286. In some embodiments, the exercise information may be transmitted to the local leaderboard 1286 locally, or not over the Internet. In this manner, collection and sorting of the information for the local leaderboard 1286 may occur much faster by avoiding delays caused by the Internet access bandwidth. Furthermore, preparing a local leaderboard 1286 may reduce the preparation time by reducing the amount of data to be collected and sorted. In this manner, an exercise group of networked exercise devices 1204 on an exercise device network 1200 may create and interact with a local leaderboard 1286, which may be updated in real-time, thereby providing the users with immediate feedback regarding their exercise information.

In some embodiments, the local leaderboard 1286 may retrieve data from the remote server 1206. For example, the local leaderboard 1286 may be prepared locally (e.g., on a computing device located within the exercise device network 1200) based on the exercise information collected by the networked exercise devices 1204. The local leaderboard 1286 may collect some leaderboard information from the remote server 1206, such as global leader exercise information. In some embodiments, information for the local leaderboard 1286 may be pre-fetched from the remote server 1206 or may be included as part of the exercise program. By only collecting some leaderboard information from the remote server 1206, the local leaderboard 1286 may reduce the amount of exercise information transmitted over the local Internet connection.

In some embodiments, the local leaderboard 1286 may collect information from the remote server 1206 based on the available Internet bandwidth. For example, if no, or very little, bandwidth is available, then the local leaderboard 1286 may not collect any information from the remote server 1206. The local leaderboard 1286 may determine how much bandwidth is available and request an appropriate amount of information from the remote server 1206. This may help to provide some outside exercise information, while allowing the leaderboard 1286 to be updated in real-time.

In some embodiments, the local leaderboard 1286 may be prepared by an exercise device 1204. For example, a lead exercise device 1204 may be identified (such as an exercise device 1204 used by a trainer or other group leader), and the lead exercise device 1204 may prepare the local leaderboard 1286 based on instructions from the group leader. In some embodiments, the local leaderboard 1286 may be prepared by any exercise device 1204 in the exercise device network 1200. In some embodiments, the local leaderboard 1286 may be a shared document, and may be updated by each of the exercise devices 1204 in the exercise device network 1200 (similar to the exercise information file 846 of FIG. 8). In some embodiments, the local leaderboard 1286 may be prepared by a remote computing device, such as a local server or network manager.

### INDUSTRIAL APPLICABILITY

This disclosure generally relates to devices, systems, and methods for creating a peer to peer (P2P) network between exercise devices. When a user selects a new exercise program to perform on an exercise device, the exercise device may look for the exercise program in the memory of other exercise devices connected to the network. If another connected exercise device has the exercise program stored in memory, then the connected exercise device may transmit the exercise program to the user's exercise device over the local network. In this manner, the number of times that the exercise program is downloaded from a cloud server may be decreased. This may increase the download speed to the user's exercise device and/or reduce server fees caused by the transmission of the exercise program over the Internet.

Using the P2P network, a user may experience a seamless transition between different exercise devices in the same workout. Each exercise device may track exercise data about the user, such as distance, heart rate, calories, time exercising, transition times, and so forth. The exercise devices may communicate the exercise data between each other. The exercise network may further prepare upcoming exercise devices for the user to use next. The user's next scheduled exercise device to use may be indicated to the user, and the next exercise device reserved for the user's use. This may reduce the amount of unnecessary and unwanted downtime during the exercise program. This may improve the exercise experience by providing a seamless transition between exercises.

In some embodiments, an exercise device network includes a local network. The local network includes two or more interconnected exercise devices. In some embodiments, the exercise devices may include any type of exercise device. For example, the exercise devices may include a treadmill, a stationary bicycle, an elliptical, a rower, a cable resistance trainer, a weight machine, free weights, smart weights (e.g., free weights including one or more sensor connected to the local network) any other type of exercise device, and combinations thereof.

In some embodiments, each exercise device in the local network may be the same type of exercise device. For example, each exercise device may be a treadmill. In some embodiments, each exercise device in the local network may be a different type of exercise device. For example, a first exercise device may be a treadmill, a second exercise device may be a stationary bicycle, a third exercise device may be a rower, and a fourth exercise device may be a set of smart weights. In some embodiments, the local network may include multiple exercise devices. For example, the first exercise device and the second exercise device may be treadmills, and the third exercise device and the fourth exercise device may be stationary bicycles. In some embodiments, the local network may include any combination of exercise device types.

While, in the embodiment shown, the local network includes four exercise devices, it should be understood that the local network may include any number of exercise devices, including 2, 3, 4, 5, 6, 7, 8, 9, 10, 15, 20, 25, 30, 40, 50, 75, 100, or more exercise devices. For example, the local network may be part of a commercial gym, and the exercise devices may be all be part of the same gym. In some examples, the local network may be part of a home gym, and the exercise devices may all be part of the user's home gym. In some examples, the local network may include two or more home gyms connected over the Internet. Connecting multiple home gyms over the Internet may reduce the amount of data transferred over a remote server, thereby reducing any data fees associated with access to the remote server.

In some embodiments, the exercise devices may be connected over a local area network (LAN). In some examples, the exercise devices may be wirelessly connected over a wireless network, such as a Wi-Fi network (e.g., IEEE 802.11 family of standards). In some examples, the exercise devices may be connected wirelessly in another manner, such as via Bluetooth, Z-wave technology, Zigbee protocol, any other wireless connection, and combinations thereof. In some examples, the LAN may include one or more Internet connections. In some examples, the LAN may include one or more Internet connections that do not connect to a remote server. In some examples, the exercise devices may be connected over a wired connection, such as an ethernet cable.

In accordance with embodiments of the present disclosure, the exercise devices may be in communication with each other. For example, the exercise devices may send and receive information between each other. In some embodiments, the exercise devices may send and receive information over the local network. In some embodiments, the exercise devices may send and receive information over the local network without the information being uploaded and/or downloaded from a remote server. In some embodiments, the exercise devices may send and receive information over the local network without the information being uploaded and/or downloaded from the remote server. In some embodiments, the exercise devices may send and receive information over the local network without sending the information over the Internet. Put another way, information may be transferred across the local network without a remote server negotiating the communication. Data transfer may happen without an intermediate server. For example, access to a remote server may be controlled by a single router or access point. All data transfer with the remote server may be controlled through the single router or access point. In this manner, a local network may transfer information between devices within the local network without transmitting information through the single router or access point.

In some embodiments, the remote server may be physically close but not connected to the local network. In some embodiments, the remote server may be physically distant from the local network. For example, the remote server may be located in a different city, in a different state/province, in a different country, in a different continent, or any other remote location. In some embodiments, data transfer across geographical boundaries (cities, states/provinces, countries, continents) may increase access costs to the remote server and/or decrease data file transfer feed. In this manner, transmitting information (such as exercise programs) across the local network (e.g., not from the remote server) may help to decrease access costs and increase transmission speed of information across the local network.

In some embodiments, the local network may be considered a peer to peer (P2P) network. A P2P network may be any network in which the computing elements of exercise devices are in communication with other computing elements of exercise devices within the same network. Exercise devices within the same P2P network may communicate with and transfer data between computing devices. In some embodiments, each exercise device within a P2P network may be in direct communication with each other exercise device. In some embodiments, two exercise devices may be in indirect communication with each other, such as in an indirect mesh network. For example, the first exercise device may be in communication with the fourth exercise device indirectly through the third exercise device. Thus, the fourth exercise device may transmit information to the first exercise device by first transmitting the information to the third exercise device, and the third exercise device may then transmit the information to the first exercise device. Thus, the third exercise device may act as a relay between the fourth exercise device and the first exercise device.

The local network may be connected to the remote server over the Internet. The remote server may include a database of exercise programs. Each of the exercise devices may be in communication with the remote server. When preparing to exercise, a user may select an exercise program from a list of available exercise programs to perform on the first exercise device. Conventionally, if the selected exercise program is not stored locally on the first exercise device, then the first exercise device may download the exercise program from the remote server. A second user may desire to perform the same exercise program on the second exercise device. Conventionally, if the selected exercise program is not stored locally on the second exercise device, then the second exercise device may download the exercise program from the remote server. This multiple downloading of the exercise program may increase the total amount of data downloaded from the remote server, resulting in increased server fees.

In accordance with embodiments of the present disclosure, when a user requests to perform an exercise program on the first exercise device, if the exercise program is not stored locally, then the first exercise device may search for the exercise program on the local network before downloading the exercise program from the remote server. If another exercise device has the exercise program stored locally, then the other exercise device may transmit the exercise program to the first exercise device over the local network. In this manner, the first exercise device may receive the exercise program from a source other than the remote server.

In accordance with embodiments of the present disclosure, transmitting exercise programs across the local network may reduce the amount of data transferred from the remote server, thereby reducing server data fees. Furthermore, some gyms may have limited bandwidth to connect to the Internet. Thus, downloading exercise programs from the remote server may take a while. This may delay the start of the user's workout, making the user wait before exercise and decreasing the exercise experience. Transferring exercising programs across the local network may increase transfer speed, thereby decreasing the user's wait time to begin exercising.

In some embodiments, each exercise device may have an active mode and a standby mode. The standby mode may be a low-power mode in which the exercise device uses less than 1 W of power. In some embodiments, transferring information (such as exercise programs) across the local exercise device network may utilize less than 1 W of power. Thus, networked exercise devices may transfer information without coming out of standby mode, or while remaining in a low-power mode. In some embodiments, an exercise device may remain in low power mode to respond to queries regarding which exercise programs are stored in its local memory. If the exercise device receives a request to transfer a particular exercise device, the exercise device may be “woken up” out of low power mode with a ping or other request to transfer the requested exercise program. This may allow more exercise devices to remain on standby, thereby reducing power requirements and reducing the risk of an electrical fire or other electrical complication.

In some embodiments, an exercise device network includes a local network having two or more interconnected exercise devices. The local network shown further includes a local server. The local server may be in communication with one or more of the exercise devices. In some embodiments, the local server may be in communication with each exercise device.

The local network may be in communication with a remote server over the Internet. The remote server may include a database of exercise programs. In some embodiments, the local server may be in communication with the remote server. The remote server may transmit one or more of the exercise programs to the local server.

In some embodiments, when a user requests an exercise program to use on an exercise device, if the exercise program is not stored on the exercise device, the exercise device may send a request to the local server for the exercise program. In some embodiments, the local server may then send the exercise program to the user's exercise device.

In some embodiments, each of the exercise devices may be in communication with the local server. In this manner, the exercise devices may send the request for the exercise program to the local server, and the local server may manage the transfer of the exercise program to the user's exercise device. In some embodiments, the local server may store the exercise program on memory included as part of the hardware of the local server. When the user requests the exercise program, the local server may transfer the exercise program from the memory on the local server to the user's exercise device.

In some embodiments, the local server may have a database of which exercise programs are stored on each exercise device. When the user requests the exercise program, the local server may identify which exercise device has a copy of the exercise program stored on the local exercise device memory. In some embodiments, the local server may provide an instruction to the exercise device storing the exercise program (e.g., the storage exercise device) to transmit the exercise program to the user's exercise device. In some embodiments, the local server may provide an instruction to the user's exercise device to request the exercise program directly from the storage exercise device. In some embodiments, the storage exercise device may transmit the exercise program to the local server and the local server may transmit the exercise program to the user's exercise device.

In some embodiments, each exercise program on the local network may be stored on the local server. The local server may have a large amount of storage and may be able to store a large number of exercise programs. Whenever a new exercise program becomes available, the local server may receive the new exercise program from the remote server at a convenient time. For example, the local server may be a computing device owned by a commercial gym. The commercial gym may have limited Internet bandwidth when the gym is open. However, after closing, Internet bandwidth may become available, and the local server may request and/or receive any new or requested exercise programs from the remote server.

In some embodiments, the local server may be representative of a home gym. For example, the local server may be a personal computing device that is connected or connectable to the exercise devices. The personal computing device may provide an interface between the exercise devices and the remote server. For example, a user may schedule a workout on one or more of the exercise devices using the personal computing device. In some embodiments, the exercise devices may not be connected to the Internet, and the personal computing device may relay an exercise program between the remote server and the exercise device. In some embodiments, the personal computing device may download one or more exercise programs and control elements of the exercise device during implementation of the downloaded exercise program. In some embodiments, the personal computing device may be a mobile phone, such as a smartphone. The display of the personal computing device may be used as a display for the exercise device.

In some embodiments, the local server may be encrypted. Encrypting the local server may help to improve the users' data privacy. In some embodiments, the local server (or any device or person on the local network) may not have the decryption key. Therefore, even if there is an unauthorized access to the local server, the information stored on the local server may not be accessed.

In some embodiments, the exercise devices may only be in communication with the local server and may not be connected to the Internet. This may help to prevent the number of requests to the remote server and reduce the amount of data downloaded from the remote server, thereby reducing data fees at the remote server. In some embodiments, the exercise devices may be connected to the remote server over the Internet. When the user requests the exercise program, the local server may determine whether the exercise program is stored on the local network. If the exercise program is stored on the local network, then the local server may manage transmission of the exercise program to the exercise device. If the exercise program is not stored on the local network, then the local server may download the exercise program from the remote server and then transfer the exercise program to the user's exercise device. In some embodiments, the local server may instruct the user's exercise device to download the exercise program from the remote server. In some embodiments, the local server may instruct the remote server to push the exercise program to the user's exercise device.

In some embodiments, a network exercise device includes local storage. The local storage may include one or more exercise programs. A user may use the network exercise device and request to perform an exercise program. In some embodiments, the local storage may include a list of available exercise programs. In some embodiments, the available exercise programs may include a listing of all the exercise programs stored on the local storage. In some embodiments, the available exercise programs may include a listing of every exercise program available to the user, either locally stored, stored on an exercise device in the local network, and/or stored on a remote server. In some embodiments, the list of available programs may include an indication of the storage location of each of the exercise programs.

In some embodiments, the user may select an exercise program that is not stored in the local storage. In some embodiments, a network storage manager may determine where the selected exercise program is stored. For example, the network storage manager may utilize a network communicator to communicate with other network exercise devices to determine if the exercise program is located on the local network. If the exercise program is located on a network exercise device connected to the local network, the network communicator may receive the exercise program from the connected network exercise device. If the exercise program is not located on the local network, then the network storage manager may send a request for the exercise device from the remote server, and the network communicator may receive the exercise device from the remote server.

In some embodiments, an exercise device network may include a plurality of exercise devices, each having a local storage. The local storages may store one or more exercise programs. In some embodiments, the local storages may store a set of exercise programs. For example, a first local storage may include a first set of exercise programs and a second local storage may include a second set of exercise programs. In some embodiments, a given set of exercise programs on an exercise device may include at least one exercise program not included on the local storage of another exercise device. For example, the first set of exercise programs stored on the first local storage includes the first exercise program and the second exercise program. The second set of exercise programs stored on the second local storage includes the third exercise program. The first set of exercise programs does not include the third exercise program, and the second set of exercise programs does not include the first exercise program and the second exercise program.

Each of the exercise devices may be in direct communication with each other over a local network. In some embodiments, when the exercise devices are in direct communication with each other over the local network, the exercise devices may communicate with each other without sending data over the Internet, or without a remote server negotiating the communication, or without a server negotiating the communication.

The local storages may have a storage capacity. While the storage capacity may be considered a number of “slots,” or number of available storage locations used to store exercise programs, it should be understood that the local storages have a storage capacity based on the available amount of memory, which may be expressed as an amount of MB, GB, TB, or more. For simplicity of illustration, it is assumed that each exercise program is the same size, and therefore, based on the size of the exercise program and the available memory, each exercise device may have a number of slots for storing exercise programs. However, it should be understood that the principles of the present disclosure may be applied to analyzing the available memory of the exercise devices and the size of the exercise programs (which may be variable between different exercise programs) to determine if there is room in the exercise device 404 for the exercise program.

In accordance with embodiments of the present disclosure, if the selected exercise program is not located on local storage, the exercise program may be downloaded to the first exercise device. However, if the first exercise device has no available storage, then one or more stored exercise programs may be deleted or removed from the local storage of the exercise device.

In accordance with embodiments of the present disclosure, when a user wishes to perform a first exercise program on a first exercise device, the user may select the first exercise program and the first exercise device may implement the first exercise program stored locally on the first local storage. However, if the user wishes to perform the third exercise program, the first exercise device may receive the third exercise program stored in the second local storage of the second exercise device. When the first exercise device may receive and store the third exercise program on the first local storage. After the third exercise program is stored on the first local storage, the first exercise device may implement the third exercise program recently stored in the first local storage. Thus, as may be seen, the user may access any of the exercise programs stored on any of the exercise devices, including the first exercise program, the second exercise program, the third exercise program, the fourth exercise program, the fifth exercise program, the sixth exercise program, and combinations thereof.

In some embodiments, an exercise program may be stored on a type of exercise device for which the exercise program is designed. For example, the exercise program may be a treadmill exercise program, and the exercise program may be stored on a treadmill. In some embodiments, the exercise program may be stored on a type of exercise device for which the exercise program is not designed. For example, the exercise program may be a rower exercise program, and the exercise program may be stored on a stationary exercise cycle. By storing exercise programs on exercise devices for which the exercise program is not designed, the storage of the entire exercise device network may be optimized.

In some embodiments, the exercise program may be transferred to the requesting exercise device from a single seed exercise device. In some embodiments, the exercise program may be stored on multiple exercise devices, and the exercise program may be transferred to the requesting exercise device from multiple seed exercise devices. Multiple seed exercise devices may increase the rate of transfer of the exercise program, allowing it to be downloaded to the requesting exercise device quickly and efficiently. For example, to transfer the exercise program, the exercise program may be split into multiple different portions or packets. Transferring the exercise program from multiple seed devices may transfer one portion (e.g., a first packet) from a first seed exercise device and a second portion (e.g., a second packet) from a second exercise device. In this manner, the exercise program may be transferred faster and more reliably by transmitting the exercise device from multiple seed exercise devices.

In some embodiments, the requested exercise program may be completely transferred between exercise devices over the exercise device network. Put another way, the requested exercise program may be transferred between exercise devices over the exercise device network without transferring any data over the Internet. In some embodiments, a program percentage (e.g., amount transferred divided by total file size) of the exercise program may be transferred over the Internet. In some embodiments, the program percentage may be in a range having an upper value, a lower value, or upper and lower values including any of 10%, 5%, 2.5%, 1%, 0.5%, 0.25%, 0.1%, 0.01%, or any value therebetween. For example, the program percentage may be greater than 0.01%. In another example, the program percentage may be less than 10%. In yet other examples, the program percentage may be any value in a range between 0.01% and 10%. In some embodiments, it may be critical that the program percentage is less than 1% to reduce bandwidth requirements and server access fees.

In some embodiments, an internet download size is the amount of information transferred over the Internet to download the exercise program. In some embodiments, the internet download size may be in a range having an upper value, a lower value, or upper and lower values including any of 1 kilobyte (kb), 10 kb, 50 kb, 100 kb, 500 kb, 1 Megabyte (Mb), 5 Mb, 10 Mb, 50 Mb, 100 Mb, or any value therebetween. For example, the internet download size may be greater than 1 kb. In another example, the internet download size may be less than 100 Mb. In yet other examples, the internet download size may be any value in a range between 1 kB and 100 Mb. In some embodiments, it may be critical that the interne download size is less than 1 Mb to reduce bandwidth requirements and server access fees.

In some embodiments, the local storage of an exercise device may be full. For example, the third exercise device may have a third local storage that is full, meaning that there is not enough remaining memory to download an entire exercise program. For example, while there may be remaining storage on the third local storage, there may not be sufficient remaining memory to download the entire selected exercise program. In some embodiments, there may be sufficient remaining memory to download other exercise programs, but not the selected memory program.

When the user selects an exercise program to be used on the third exercise device, such as the second exercise program, the exercise device may determine whether there is sufficient memory in the third local storage to download the second exercise program. If there is not sufficient memory in the third local storage, then, as discussed in further detail herein, one or more of the downloaded exercise programs stored on the third local storage (e.g., the first exercise program, the fourth exercise program, the fifth exercise program, or the sixth exercise program) may be deleted from the third local storage. This may free up storage capacity on the third local storage and allow the second exercise program to be downloaded to the third local storage.

In some embodiments, a method for implementing an exercise program, may include receiving an input to perform an exercise program. In some embodiments, the input may be received at the exercise device to be used. In some embodiments, the input may be received from a remote device. For example, the input may be received at an exercise manager, or a local server connected to each of the exercise devices. For example, the exercise devices on the exercise network may be connected to a mobile device over the Internet. The user may plan to perform an exercise on a specific exercise device. The user may select the exercise program to be performed and the exercise device on which to perform the exercise program. In some embodiments, the user may “reserve” the exercise device for a specific period of time to perform the exercise program.

After the input is received, the exercise device may determine whether the data file of the exercise program is located in the exercise device network. For example, the exercise device (or local server, or remote server), may search the local storage of each exercise device for whether the exercise network includes the data file of the exercise program. In some embodiments, determining whether the exercise program data file is located on the exercise network may include querying the exercise network and/or each exercise device on the exercise network for the exercise program. Querying the exercise network may help to determine which, if any, of the exercise devices on the exercise network has stored the exercise program on local storage. If one of the exercise devices on the exercise network has stored the exercise program on local storage, the exercise device may provide a positive response to the query. If an exercise device does not have the exercise program stored, it may remain silent or provide a negative response.

In some embodiments, determining whether the data file is located on the exercise network may include checking an exercise program database stored on the local network. The exercise program database may include a listing of which exercise programs are stored on which exercise devices on the local network. The requesting exercise device may check the exercise program database to determine which exercise device(s) have stored the exercise program. The requesting exercise device may then provide an individual request for the exercise program to the exercise device(s) which has the exercise program stored. In some embodiments, a local server may have the exercise program database stored, and determining whether the data file is located on the exercise network may include querying the local server. The local server may provide a response regarding which exercise device(s) (if any) are storing the exercise program.

If the data file is located on the exercise network, then the exercise program may be transferred between exercise devices on the exercise device network to the user's exercise device. If the data file is not located on the exercise network, then the exercise program may be downloaded from the cloud. This may help to prevent multiple downloads of the exercise program to devices on the exercise device network, thereby reducing data fees on the cloud.

In some embodiments, an exercise device network may include a plurality of exercise devices, each having a local storage. The exercise device network shown includes a first exercise device having a first local storage, a second exercise device having a second local storage, a third exercise device having a third local storage, and a fourth exercise device having a fourth local storage. The local storages may store one or more exercise programs. The exercise programs may include a first exercise program, a second exercise program, a third exercise program, a fourth exercise program, a fifth exercise program, a sixth exercise program, a seventh exercise program, an eighth exercise program, a ninth exercise program, and a tenth exercise program. Each of the exercise devices may be in communication with each other.

In accordance with embodiments of the present disclosure, when a user wishes to perform a first exercise program on a first exercise device, the user may select the first exercise program and the first exercise device may implement the first exercise program stored locally on the first local storage. However, if the user wishes to perform the third exercise program, the third exercise program may need to be transferred to the first local storage.

In the embodiment shown, the first local storage may be full (e.g., the first local storage may not have sufficient remaining storage to download another exercise program). Thus, to install the third exercise program on the first exercise device, one or more of the exercise programs stored on the first local storage may need to be deleted. In some embodiments, one or more of the exercise programs on the first local storage may be deleted. In some embodiments, deleting an exercise program may include wiping the exercise program from the local storage. In some embodiments, deleting the exercise program may include marking the memory allocated to the exercise program for over-writing or replacement.

In some embodiments, deleting the exercise program may include transferring the exercise program to the local storage of a different exercise device. For example, the exercise program may not be stored on any other local storage of any exercise device. To maintain the exercise program on the exercise device network, the exercise program may be transferred to another exercise device before being deleted from the local storage.

In some embodiments, one or more exercise programs may be flagged for deletion. For example, the user may flag one or more exercise programs for use. For example, the user may identify or select one or more exercise programs to be deleted. The user may identify or select the one or more exercise programs to be deleted based on any reasoning. For example, the user may desire to perform a new exercise program every time he or she exercises, the user may feel that he or she has performed a particular exercise program enough times, the user may arbitrarily identify an exercise program for deletion, any other reasoning, and combinations thereof. In some embodiments, the user may flag an exercise program for deletion based on a prompt from the exercise device. For example, when the user selects an exercise program not located on the exercise device, the exercise device (or other input device) may prompt the user to select an exercise program for deletion.

In some embodiments, the exercise device may identify or flag one or more exercise programs for deletion or replacement. In some embodiments, the exercise device network may flag an exercise program based on network-wide metrics. For example, the exercise device may identify low-utilization exercise programs (e.g., the least-used exercise programs) for deletion. In some embodiments, the low-utilization exercise program may have the lowest number of uses when a new exercise program is selected for download. In some embodiments, the least-used exercise program may have the longest time between last use when a new exercise program is selected for download. In some embodiments, the low-utilization exercise program may have the lowest user rating of all the exercise programs on the exercise device. In some examples, the exercise device may flag for deletion the exercise program that has been on the exercise device the longest. In some examples, the exercise device may flag for deletion the exercise program that has the largest data file size. In some examples, the exercise device may flag for deletion the exercise program that has the smallest data file size for which a deletion may provide room on the local storage for the selected exercise program.

In some embodiments, the exercise device network may identify or flag one or more exercise programs for deletion or replacement. For example, the exercise device network may identify or flag an exercise program for deletion if there are one or more copies of the exercise program on other exercise devices within the exercise device network. In some examples, the exercise device network may identify or flag the least-used exercise programs across the entire exercise device network. For example, despite the first exercise device having run the second exercise program multiple times, the total usage of the second exercise program on the other exercise devices of the exercise device network may be low, resulting in the second exercise program being flagged for deletion.

In some embodiments, the exercise device network may receive instructions from the cloud to identify or flag one or more exercise programs for deletion. For example, a specific exercise program may have discontinued cloud support, may have low overall usage across multiple exercise device networks, and may be flagged for replacement by more highly rated or newer exercise programs.

In some embodiments, when the user selects an exercise program to download onto an exercise device, the exercise device may delete or replace the flagged exercise program with the selected exercise program. For example, the user may desire to perform the fourth exercise program, which is stored on the third exercise device, on the first exercise device. Upon making the selection, the first exercise device may delete one or more flagged exercise programs (e.g., the second exercise program and/or the tenth exercise program) to make room for the fourth exercise program.

In some embodiments, one or more exercise programs may be starred or otherwise identified (e.g., flagged, marked) to be retained (e.g., be prevented from deletion). When a user selects an exercise program that is not located on an exercise device, the exercise device may not delete the starred exercise program. In some embodiments, the user may star an exercise program. For example, the user may star an exercise program because he or she particularly likes the exercise program, the trainer, the exercise device, and combinations thereof.

In some embodiments, the exercise device may star an exercise program. For example, the exercise device may star an exercise program that has the most uses, the most recent uses, the highest user reviews, and combinations thereof. In some examples, the exercise device may star exercise programs that have been recently downloaded. Put another way, the exercise device may star exercise programs that have been downloaded within a period of time, such as an hour, a day, a week, a month, and so forth.

In some embodiments, the exercise device network may star one or more exercise programs. In some embodiments, the exercise device network may star an exercise program based on network-wide metrics. For example, the exercise device network may star an exercise program based on network-wide high usage or high user rating, based on how recently it was downloaded to the network or for any other reason, and combinations thereof. In some embodiments, the cloud may instruct the exercise device network to star an exercise program. For example, the cloud may instruct the exercise device network to star exercise programs that are popular across multiple networks, that are newly released, promotional exercise programs, seasonal exercise programs, for any other reason, and combinations thereof.

In the embodiment shown, the fourth exercise device includes the first exercise program and the fifth exercise program. The first exercise program is shown as starred. Thus, if a user wishes to implement an exercise program on the fourth exercise device, the fifth exercise program may be deleted and the new exercise program downloaded.

When planning a workout, the user may be presented with a list of available exercise programs. The list of available exercise programs may include indications regarding on which exercise device a particular exercise program is located. This may provide an indication to the user regarding how fast a particular exercise program may be loaded onto the exercise device. For example, if a user wishes to perform the third exercise program on the first exercise device, the list of available exercise programs may indicate that the third exercise program is located on an exercise device in the exercise network. This may indicate to the user that the third exercise program may be transferred to the first exercise device (e.g., from the second exercise device) relatively quickly. However, if the user wishes to perform an exercise program not located on any device within the exercise device network, the user may infer that downloading the exercise program from the cloud to the first exercise device may take longer.

In some embodiments, the cloud server may push one or more updated exercise programs to one or more exercise devices on the exercise device network. For example, a new exercise program may be released. The cloud server may push the newly released exercise program to the exercise device network. In this manner, the cloud server may control when a particular exercise device network may receive the new exercise program. This may help to balance the data traffic load on the cloud server.

In some embodiments, an exercise device network may have limited access to the Internet. For example, the exercise device network may have limited Internet bandwidth, data caps, or other limitations on the Internet access. In some embodiments, a new exercise program may be downloaded to an exercise device during low Internet access or usage. For example, access to the Internet is usually lower at night. To preserve daytime bandwidth, the cloud server may push a new exercise program to the exercise device network at night. In some embodiments, the exercise device network may request or download the new exercise program from the cloud server at night.

In some embodiments, the user may schedule to perform an exercise program for a future time. The future time may be minutes, hours, days, or weeks in the future of when the user makes a selection of an exercise program. If the user schedules to perform the exercise program in the future, the exercise device and/or the exercise network may schedule the download of the exercise program for a time that optimizes network bandwidth. This may free up Internet bandwidth for the exercise device network, thereby allowing increased user access (e.g., for music/video streaming) and/or increased gym access (e.g., for exercise information up/download).

Different exercise devices may have different internal storage capacities. In some embodiments, a managing server of the exercise device network may manage the storage of the different exercise devices. The managing server may flag or star different exercise programs, manage downloading of new exercise programs, perform any other management task, and combinations thereof. In some embodiments, an exercise device network may include a local storage device, such as a computer, external hard drive, local server, and so forth. The local storage device may maintain a library of exercise programs. When a user wishes to perform an exercise program not stored on a particular exercise device, the local storage device may transfer the selected exercise program to the exercise device over the local area network for the exercise device network.

In some embodiments, a method for implementing an exercise program may include receiving an input to perform an exercise program. After the input is received, the exercise device may determine whether the data file of the exercise program is located on an exercise device of the exercise device network. If the data file is located on an exercise device of the exercise device network, the exercise program may be transferred between exercise devices over the exercise device network.

If the data file is not located on the exercise device, the exercise device may determine whether there is storage available on the local storage of the exercise device. If storage is available on the local storage, then the exercise program may be downloaded to the exercise device from the cloud server. If storage is not available on the local storage, then one or more stored exercise programs stored on the exercise device may be deleted from the local storage. The selected exercise program may then be downloaded from the cloud.

In some embodiments, a user may wish to perform a single workout on multiple exercise devices. For example, an exercise program may include a first portion that is to be performed on a first exercise device, such as a treadmill, and a second portion that is to be performed on a second exercise device. The user may desire to track exercise information while performing the exercise. Exercise information may include heartrate, time, calories burned, speed, distance, steps, cadence, any other exercise information, and combinations thereof.

In some embodiments, the user may wish to collect information that is continuous across both the first portion and the second portion of the exercise program. For example, the user may wish to have an average heartrate, total time, total calories burned, average speed, total distance, total steps, average cadence, any other total or average exercise information, and combinations thereof, that is cumulative or averaged across the entire workout.

Because the workout includes portions on both the first exercise device and the second exercise device, each exercise device may collect exercise information using one or more exercise sensors. The exercise sensors may include any exercise sensor, including heart rate sensors, speed sensors, pressure sensors, weight sensors, any other sensor, and combinations thereof. In some embodiments, the exercise devices may track the collected exercise information using one or more exercise information tracker. In some embodiments, each exercise device may store the exercise information on local storage. For example, the first exercise device may collect exercise information using a first exercise sensor and track the exercise information on a first local storage using a first exercise information tracker. In some examples, the second exercise device may collect exercise information using a second exercise sensor and track the exercise information on a second local storage using a second exercise information tracker.

In some embodiments, the exercise devices may track cumulative and/or average exercise information on a combined exercise information file. The exercise devices may be connected to each other over the exercise device network. In some embodiments, the first exercise device may be connected to the second exercise device over the exercise device network using a first network communicator. The second exercise device may be connected to the first exercise device over the exercise device network using a second network communicator. In this manner, each exercise device may modify the exercise information file over the exercise device network. In some embodiments, each exercise device may modify the exercise information file without transferring any information over the Internet. For example, all of the collected information may be transferred over the exercise device network.

In some embodiments, the combined exercise information file may be stored remote from the exercise devices. For example, the combined exercise information file may be stored on a local server or computer. When the user performs a first portion of the exercise program on the first exercise device, the first exercise device may collect the exercise information with the first exercise sensor and record the exercise information on the exercise information file using the first exercise information tracker via the first network communicator. When the user performs a second portion of the exercise program on the second exercise device, the second exercise device may collect the exercise information with the second exercise sensor and record the exercise information on the exercise information file using the second exercise information tracker via the second network communicator.

In some embodiments, the combined exercise information file may be stored on the first local storage. The first exercise device may record the exercise information on the exercise information file directly on the first local storage using the first exercise information tracker. The second exercise device may record the exercise information on the exercise information file on the first local storage using the second exercise information tracker and the second network communicator.

In some embodiments, the combined exercise information file may be stored on the second local storage. In some embodiments, the second exercise device may record the exercise information on the exercise information file directly on the second local storage using the second exercise information tracker. The first exercise device may record the exercise information on the exercise information file on the second local storage using the first exercise information tracker and the first network communicator. In some embodiments, the combined exercise information file may be stored on each exercise device. Updates to the exercise information file may be transmitted to each exercise device using the network communicators.

In some embodiments, the combined exercise information file may be stored on the first exercise device during the first portion of the exercise program. After the user finishes the first portion of the exercise program, the combined exercise information file may be transmitted to the second exercise device and the combined exercise information file removed from the first exercise device. This may help to reduce the number of locations that the user's data is stored, thereby improving the user's information privacy and/or sense of information privacy.

Conventionally, a combined exercise information file may be stored on the cloud server. Thus, every update to the combined exercise information file may be uploaded to the cloud and then downloaded to the exercise device being used. This may increase the amount of network traffic over the Internet connection to remote cloud. As discussed herein, the Internet bandwidth may be limited. This may result in reduced transfer of the exercise information between exercise devices. This may further result in exercise delays as the user waits for the exercise information to be transferred to the next exercise device.

As discussed herein, updating the combined exercise information file over the exercise device network may reduce or prevent the amount of information transmitted to the cloud server over the Internet. Because the exercise devices are connected over the exercise device network, the transfer of exercise information may be fast, or instantaneous. This may help to reduce the delay between portions of the exercise program. This may improve the user experience by allowing the user to maintain his or her heartrate at a desired level when transitioning between exercise devices.

In some embodiments, updating and storing the combined exercise information file on the local exercise device network may allow the exercise device network to upload the combined exercise information file to a remote server anytime. For example, the exercise device network may upload the combined exercise information file during low-usage times of the exercise device network Internet connection. This may help to make Internet bandwidth available for other uses, such as media access, downloading of new exercise programs, and so forth.

When the user begins performing an exercise on an exercise device, he or she may log in to the exercise device using a user login. Using the user login, the user may log on to each exercise device in any manner, including a user ID and password, an RFID identifier, near-field communication identifier, biometric identifier (such as fingerprint, EKG pattern, facial recognition, voice recognition, etc.), any other login information, and combinations thereof.

While embodiments of the present disclosure have discussed an exercise device network tracking exercise information for a single user's exercise program, it should be understood that the exercise device network may track multiple user's exercise information on multiple shared exercise information files. For example, a first user may perform a portion of a first exercise program on a first exercise device, and the first exercise device may track the associated exercise information on a first shared exercise information file. When the portion is complete, the first user may move to a second exercise device, which may continue to track the user's exercise information using the first shared exercise information file. When the first user finishes using the first exercise device, a second user may begin using the first exercise device. The first exercise device may track the second user's associated exercise information on a second shared exercise information file. Thus, the exercise device network may track multiple shared exercise information files simultaneously. For example, the first exercise device may update the second shared exercise information file while the second exercise device is simultaneously updating the first shared exercise information file.

In some embodiments, a method for implementing an exercise program may include receiving an input to perform an exercise program. As discussed herein, the input may be received at any location. For example, a user may provide an input on an interactive display of an exercise device. In some examples, the user may provide the input on a remote computing device. For example, the user may provide the input using an app on a mobile device or other remote computing device.

The exercise program may be performed across multiple exercise devices. For example, the exercise program may include a plurality of portions. A first portion may be performed on a first type of exercise device (such as a rower) and a second portion may be performed on a second type of exercise device (such as an elliptical trainer). In some examples, the first exercise device and the second exercise device may be the same type of exercise device. For example, a user may perform the first portion on the first exercise device (such as a stationary exercise cycle). The user may dismount or otherwise leave the first exercise device after the first portion is over (such as for a rest, a restroom break, stretching, or any other reason). While the user has left the first exercise device, another user may begin using the first exercise device. When it is time to perform the second portion of the exercise program, the user may begin using the second exercise device of the same type as the first exercise device without interrupting his exercise program. By sharing exercise information across exercise devices, the user may experience greater flexibility regarding which exercise devices he or she may use. Furthermore, this may help increase exercise device utilization at a gym, especially during peak usage time, by reducing exercise device downtime.

In some examples, the first portion may be performed on a first type of exercise device, and the second portion may be performed using free-weights (including free weights connected to the exercise device network). The exercise program may include a third portion that is performed on the first type of exercise device. In some embodiments, the first portion and the third portion may be performed on the same exercise device. In some embodiments, the first portion and the third portion may be performed on different exercise devices of the same type. For example, the user may perform the exercise program in a crowded gym. While the user is performing the second portion (e.g., using the free weights), a different user may begin using the first exercise device. When the user finishes the second portion, he may continue the exercise on a second exercise device of the same type as the first exercise device.

The user may perform the first portion of the exercise program on a first exercise device. While performing the first portion of the exercise program, the first exercise device may track exercise information about the user and/or the exercise program on a shared exercise information file. The user may then perform the second portion of the exercise program on a second exercise device. The second exercise device may track the exercise information related to the second portion on the shared exercise information file. The shared exercise information file may be shared between the first exercise device and the second exercise device.

In some embodiments, when the user begins performing an exercise on an exercise device, he or she may log in to the exercise device causing the exercise device to receive user login information. The user login information may be any type of user login information, including a user ID and password, an RFID identifier, near-field communication identifier, biometric identifier (such as fingerprint, EKG pattern, facial recognition, voice recognition, etc.), any other login information, and combinations thereof. After the user logs in, the exercise device may determine whether there is an active exercise program associated with the user. An active exercise program may be an exercise program that the user has selected and performed at least a portion of on another exercise device. If the exercise device determines that there is not an active exercise program associated with the user, the exercise device may prompt the user to select an exercise program to be implemented.

If the exercise device determines that there is an active exercise program associated with the user, the exercise device may continue the active exercise program. Continuing the active exercise program may include determining which portion(s) of the exercise program have been completed, and continuing the exercise program by implementing the next portion. In some embodiments, if portion of the exercise program is inconsistent with the type of exercise device, then the exercise device may prompt the user to move to the appropriate exercise device.

When continuing the active exercise program, the exercise device may further determine whether there is an active exercise information file associated with the user on the exercise device network. If there is an active exercise information file associated with the user, then the exercise device may modify the active exercise information file with exercise information relevant to the portion of the exercise program being performed. Put another way, the exercise device may track exercise information to the active exercise information file for the user. If there is not an active exercise information file associated with the user on the exercise device network, then the exercise device may create an active exercise information file for the user. The exercise device may then track exercise information to the active exercise information file.

On the exercise device network, a user may schedule an exercise program to be performed in a particular time slot of an exercise device. Reserving a single exercise program across multiple exercise devices may help a user to complete an exercise program across multiple exercise deices uninterrupted, or without waiting for an exercise device to become available.

In the embodiment shown, a first user may reserve the first exercise device from a time of between 12:00 and 12:30 to perform a first portion of a first exercise program. The first user may then reserve the second exercise device from a time of between 12:30 and 1:00 to perform a second portion of the first exercise program. If another user tries to log in to the first exercise device when the first exercise device is reserved by the first user, the first exercise device may not function for the second user. This may help a user to know that he or she has an uninterrupted time available to exercise on the first exercise device.

In some embodiments, the exercise devices may be scheduled based on the duration of individual portions of an exercise program. For example, after the user completes the first portion of the first exercise program, the second user may perform a portion of an exercise program on the first exercise device. In some embodiments, the second user may perform the portion of the exercise program while the first user is performing the second portion of the first exercise program. In this manner, the first exercise device may not be scheduled for the entire duration of the exercise program, but rather for the duration of the relevant portion of the exercise program. This may help to increase exercise device utilization, thereby increasing the capacity of the gym.

During a group exercise session, multiple users may perform the same exercise program on multiple exercise devices. Each exercise device may collect exercise information about their associated user. The collected exercise information may be different based on the different abilities and fitness levels of the different users. In some embodiments, users may wish to compare at least a portion of their exercise information with other users. For example, a user may wish to compare his or her speed, distance, time, power, and other exercise information against other users that have used the same exercise program. In some embodiments, a group of user's exercise information may be presented as a leaderboard, where the leaderboard includes a summary of relevant exercise information for various users, often sorted by speed, distance, power, and so forth.

Conventionally, leaderboard information is collected from a remote server (e.g., the cloud). Each exercise device may be connected to the remote server and may upload the relevant exercise information to the remote server, and the leaderboard may be created using the exercise information stored on the remote server. In this manner, a user may compare him or herself against remote users, including global users, national users, and local users. In some embodiments, a leaderboard of a particular exercise group may be collected from the remote server. For a large database on the remote server, the time to search, sort, and send all of the leaderboard information may become cumbersome, resulting in delays to the update of the leaderboard.

In accordance with embodiments of the present disclosure, an exercise device network may prepare a local leaderboard. The exercise information presented on the local leaderboard may be collected from the networked exercise devices connected to the exercise device network. Each exercise device may transmit the exercise information over the exercise device network to the local leaderboard. In some embodiments, the exercise information may be transmitted to the local leaderboard locally, or not over the Internet. In this manner, collection and sorting of the information for the local leaderboard may occur much faster by avoiding delays caused by the Internet access bandwidth. Furthermore, preparing a local leaderboard may reduce the preparation time by reducing the amount of data to be collected and sorted. In this manner, an exercise group of networked exercise devices. on an exercise device network may create and interact with a local leaderboard, which may be updated in real-time, thereby providing the users with immediate feedback regarding their exercise information.

In some embodiments, the local leaderboard may retrieve data from the remote server. For example, the local leaderboard may be prepared locally (e.g., on a computing device located within the exercise device network) based on the exercise information collected by the networked exercise devices. The local leaderboard may collect some leaderboard information from the remote server, such as global leader exercise information. In some embodiments, information for the local leaderboard may be pre-fetched from the remote server or may be included as part of the exercise program. By only collecting some leaderboard information from the remote server, the local leaderboard may reduce the amount of exercise information transmitted over the local Internet connection.

In some embodiments, the local leaderboard may collect information from the remote server based on the available Internet bandwidth. For example, if no, or very little, bandwidth is available, then the local leaderboard may not collect any information from the remote server. The local leaderboard may determine how much bandwidth is available and request an appropriate amount of information from the remote server. This may help to provide some outside exercise information, while allowing the leaderboard to be updated in real-time.

In some embodiments, the local leaderboard may be prepared by an exercise device. For example, a lead exercise device may be identified (such as an exercise device used by a trainer or other group leader), and the lead exercise device may prepare the local leaderboard based on instructions from the group leader. In some embodiments, the local leaderboard may be prepared by any exercise device in the exercise device network. In some embodiments, the local leaderboard may be a shared document, and may be updated by each of the exercise devices in the exercise device network. In some embodiments, the local leaderboard may be prepared by a remote computing device, such as a local server or network manager.

Following are embodiments of sections of the present disclosure:
A1. A method for implementing an exercise program, comprising:
   receiving an input on a first exercise device of a plurality of exercise devices to perform the exercise program, wherein the plurality of exercise devices are connected over a local network;
   determining whether the exercise program is located on a second exercise device of the plurality of exercise devices; and
   if the exercise program is located on the second exercise device, transferring the exercise program to the first exercise device from the second exercise device.
A2. The method of section A1, wherein the exercise program is transferred to the first exercise device over the local network.
A3. The method of section A1, wherein the exercise program is transferred over the local network without a remote server negotiating communication between the first exercise device and the second exercise device.
A3. The method of section A1, wherein the exercise program is transferred to the first exercise device without being uploaded or downloaded over the Internet.
A4. The method of section A1, wherein determining whether the exercise program is located on the second exercise device includes querying each exercise device of the plurality of exercise devices to determine which exercise device of the plurality of exercise devices is storing the exercise program.
A5. The method of section A1, wherein determining whether the exercise program is located on the second exercise device includes querying an exercise program database of exercise programs stored on each exercise device of the plurality of exercise devices.
A6. The method of section A1, wherein the local network is a local area network, and the exercise devices are connected wirelessly over the local area network.
A7. The method of section A1, wherein transferring the exercise program to the first exercise device includes transferring the exercise program from multiple exercise devices of the plurality of exercise devices.
A8. The method of section A1, further comprising, if the exercise program is not located on any exercise device of the plurality of exercise devices, downloading the exercise device from a remote server.
A9. The method of section A1, further comprising, if the first exercise device of the plurality of exercise devices has no available storage, removing a stored exercise program from the first exercise device of the plurality of exercise devices.
A10. The method of section A9, wherein the stored exercise program is a low-utilization exercise program.
A11. The method of section A9, wherein the stored exercise program is flagged for deletion.
B1. An exercise system, comprising:
   a first exercise device, including:
      a first local storage including a first set of exercise programs; and
      a first network communicator;
   a second exercise device, including:
      a second local storage including a second set of exercise programs; and
      a second network communicator in direct communication with the first exercise device over a local network.
B2. The exercise system of section B1, wherein the first network communicator and the second network communicator are in direct communication without sending data over the Internet.
B3. The exercise system of section B1, wherein the second set of exercise programs includes at least one exercise program not included in the first set of exercise programs, and wherein the second network communicator sends the at least one exercise program directly to the first exercise device.
B4. The exercise system of section B3, wherein the second network communicator sends the at least one exercise program to the first exercise device without a remote server negotiating communication between the first exercise device and the second exercise device.
B5. The exercise system of section B3, wherein less than 1% of data sent to or received at the first exercise device when transmitting the at least one exercise program is transmitted over the Internet.
B6. The exercise system of section B3, wherein less than 1 MB of data sent to or received by the first exercise device when transmitting the at least one exercise program is transmitted over the Internet.
B7. The exercise system of section B1, wherein the first exercise device is a different type of exercise device than the second exercise device.
C1. A method for implementing an exercise program, comprising:
   storing an exercise program on a first exercise device; and
   directly transmitting the exercise program to a second exercise device over a local network without a remote server negotiating communication between the first exercise device and the second exercise device.
D1. A method for performing an exercise, comprising:
   receiving a selection for the exercise program, the exercise program including:
      a first portion for use on a first exercise device; and
      a second portion for use on a second exercise device, wherein the first exercise device and the second exercise device are connected over a local network;
   tracking exercise data with the first exercise device during the first portion of the exercise program; and
   tracking the exercise data with the second exercise device during the second portion of the exercise program, wherein the exercise data is shared between the first exercise device and the second exercise device over the local network.
D2. The method of section D1, wherein the first exercise device is a different type of exercise device than the second exercise device.
D3. The method of section D1, further comprising presenting the exercise data on a local leaderboard.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method for implementing an exercise program, comprising:
receiving an input on a first exercise device of a plurality of exercise devices to
perform the exercise program, wherein the plurality of exercise devices
are connected over a local network;
determining whether the exercise program is located on a second exercise device of the plurality of exercise devices; and
if the exercise program is located on the second exercise device, transferring the exercise program to the first exercise device from the second exercise device.

2. The method of claim 1, wherein the exercise program is transferred to the first exercise device over the local network.

3. The method of claim 1, wherein the exercise program is transferred over the local network without a remote server negotiating communication between the first exercise device and the second exercise device.

4. The method of claim 1, wherein the exercise program is transferred to the first exercise device without being uploaded or downloaded over the Internet.

5. The method of claim 1, wherein determining whether the exercise program is located on the second exercise device includes querying each exercise device of the plurality of exercise devices to determine which exercise device of the plurality of exercise devices is storing the exercise program.

6. The method of claim 1, wherein determining whether the exercise program is located on the second exercise device includes querying an exercise program database of exercise programs stored on each exercise device of the plurality of exercise devices.

7. The method of claim 1, wherein the local network is a local area network and the exercise devices are connected wirelessly over the local area network.

8. The method of claim 1, wherein transferring the exercise program to the first exercise device includes transferring the exercise program from multiple exercise devices of the plurality of exercise devices.

9. The method of claim 1, further comprising, if the exercise program is not located on any exercise device of the plurality of exercise devices, downloading the exercise device from a remote server.

10. The method of claim 1, further comprising, if the first exercise device of the plurality of exercise devices has no available storage, removing a stored exercise program from the first exercise device of the plurality of exercise devices.

11. The method of claim 10, wherein the stored exercise program is a low-utilization exercise program.

12. The method of claim 10, wherein the stored exercise program is flagged for deletion.

13. An exercise system, comprising:
a first exercise device, including:
a first local storage including a first set of exercise programs; and
a first network communicator;
a second exercise device, including:
a second local storage including a second set of exercise programs; and
a second network communicator in direct communication with the first exercise device over a local network.

14. The exercise system of claim 13, wherein the first network communicator and the second network communicator are in direct communication without sending data over the Internet.

15. The exercise system of claim 13, wherein the second set of exercise programs includes at least one exercise program not included in the first set of exercise programs, and wherein the second network communicator sends the at least one exercise program directly to the first exercise device.

16. The exercise system of claim 14, wherein the second network communicator sends one or more of the exercise programs of the first set of exercise programs to the first exercise device without a remote server negotiating communication between the first exercise device and the second exercise device.

17. The exercise system of claim 14, wherein less than 1% of data sent to or received at the first exercise device when transmitting the one or more of the exercise programs of the first set of exercise programs is transmitted over the Internet.

18. The exercise system of claim 14, wherein less than 1 MB of data sent to or received by the first exercise device when transmitting the one or more of the exercise programs of the first set of exercise programs is transmitted over the Internet.

19. The exercise system of claim 13, wherein the first exercise device is a different type of exercise device than the second exercise device.

20. A method for implementing an exercise program, comprising:
storing an exercise program on a first exercise device; and
directly transmitting the exercise program to a second exercise device over a local network without a remote server negotiating communication between the first exercise device and the second exercise device.
